(19) United States
(12) Patent Application Publication
(10) Pub. No.: US 2026/0323777 A1
(43) Pub. Date: Oct. 15, 2026

(54) RAPID, UNIFORM AND SCALABLE REWARMING FOR CRYOPRESERVATION USING JOULE HEATING

(71) Applicant: Regents of the University of Minnesota, Minneapolis, MN (US)

(72) Inventors: Li Zhan, Medord, MA (US); Zonghu Han, Falcon Heights, MN (US); Qi Shao, Plymouth, MN (US); Michael L. Etheridge, Maple Grove, MN (US); John C. Bischof, St. Paul, MN (US)

(21) Appl. No.: 18/880,806

(22) PCT Filed: Jun. 29, 2023
(86) PCT No.: PCT/US2023/026539

§ 371 Date: Jan. 2, 2025

Related U.S. Application Data
(60) Provisional application No. 63/358,377, filed on Jul. 5, 2022.

(51) Int. Cl. H05B 3/00; A01N 1/144; C12N 5/00

(52) U.S. Cl. CPC H05B 3/0019; A01N 1/144; C12N 5/544

(57) ABSTRACT

Cryopreserved biomaterials are rewarmed with resistive heating methods. The method is scalable to large number of specimens. The cryopreserved biomaterial is in thermal contact with an electrical conductor. The electrical conductor is connected to a generator capable of discharging a turnable voltage pulse. The voltage pulse is tuned to deliver the selected heat to the conductor to rapidly and uniformly rewarm the cryopreserved biomaterial.

This invention was made with government support under OD028758 and DK117425 awarded by the National Institutes of Health. The government has certain rights in the invention.

This invention was made with government support under EEC-1941543 awarded by the National Science Foundation. The government has certain rights in the invention.

### BACKGROUND

Biological time can be effectively “stopped” when living biological systems are successfully cooled and stored at cryogenic temperatures (i.e., cryopreservation). Cryopreservation has far-reaching implications such as banking and supplying “off the shelf” biological systems for cell therapies, transplantable tissues, research model organisms, and bio-conservation.

### SUMMARY

Cryopreservation has brought massive societal and economic benefits by enabling the ability to store and transport biological systems at multiple length scales ranging from cells to embryos and tissues, but the potential for significant impact remains challenging due to the limits of existing rewarming approaches. Rapid and scalable (both in throughput and biosystem size) rewarming techniques for low cryoprotective agent (CPA) concentrations for reduced toxicity are lacking. The present description includes an innovative Joule heating based platform technology to overcome this obstacle. The biological systems can be rapidly rewarmed by thermal contact with an electrical conductor that is delivered a voltage pulse from a connected generator. The method includes tuning a voltage pulse to deliver the selected heat to the conductor, for example, based on the size of the cryopreserved biospecimen and diffusion of the heat throughout the cryopreserved biospecimen so as to achieve rapid and sufficiently uniform rewarming. The voltage pulse may be tuned with respect to, for example, magnitude, duration, and/or wave form.

In one aspect, the present description relates to a method of rewarming a cryopreserved biological composition. The method includes applying resistive heating to rewarm a cryopreserved biological composition, wherein the resistive heating includes discharging a tunable voltage pulse from an electrical generator to an electrical conductor, wherein the generator is electrically connected to the conductor. The cryopreserved biological composition includes a cryopreserved biomaterial suspended in and/or perfused with a cryoprotective solution, wherein the cryopreserved biological composition is in thermal contact with the conductor. The voltage pulse is tuned to generate heat sufficient to rewarm across a heat diffusion length of the biomaterial with minimal biomaterial damage. The heat diffusion length may be about the thickness of the biomaterial. The heat diffusion length may be about half the thickness of the biomaterial. The heat diffusion length may be between about 1 μm and about 10 mm. The voltage pulse discharged from the generator may be tuned to generate a minimum heating rate and temperature change in the cryopreserved composition sufficient to rewarm the cryopreserved biomaterial with minimal biomaterial damage. The method may further comprise determining a voltage pulse width that generates rewarming across the heat diffusion length of the biomaterial. The voltage width may be tuned to achieve a minimum heating rate and a temperature change of the cryopreserved biomaterial. The voltage pulse width may be between about the heat diffusion time of the cryopreserved composition and about 10 times the heat diffusion time of the cryopreserved composition. The voltage pulse may have a pulse width of between about 0.1 μs and about 10 seconds. The cryoprotective solution may include cryoprotective agents selected from the group consisting of dimethyl sulfoxide, glycerol, propylene glycol, ethylene glycol, formamide, sucrose, glucose, glucose analogs, trehalose, raffinose, polyvinylpyrrolidone, and combinations thereof. The conductor may include stainless steel, titanium, platinum, aluminum, zinc, gold, silver, nickel, iron, chromium, molybdenum, silicon, carbon, ceramic, doped plastic and combinations thereof. The conductor can include a conductivity and geometry, wherein the conductivity and the geometry of the conductor are selected to generate the specific absorption rate (SAR) to rewarm the biomaterial at a heating rate to minimize biomaterial damage. The geometry of the conductor may include a sheet, a mesh, a wire, a cylinder, a tube, a coil, a wound structure, a porous structure, a matrix structure, a channel and combinations thereof. The conductor configuration may be adapted to generate a sufficiently uniform heating rate across the heat diffusion length of the biomaterial to minimize biomaterial damage. The method may include an intermediate layer between the conductor and the biomaterial. The biomaterials are selected from the group consisting of adherent cells, droplets or thin films of cell suspensions, protein suspensions, embryos, oocytes, larvae, organisms, tissue slices, biopsies, isolated tissues, engineered tissues, cell clusters, organoids and the like. The voltage pulse form may be an exponentially decaying wave, sine wave, a rectangular wave, a triangular wave, a sawtooth wave or a square wave. The magnitude of the voltage pulse may be between about 1V and 10,000V. The method may be adapted for high throughput and/or larger size of the biomaterial. The biomaterial damage may be from ice crystallization causing damage to cellular and tissue structures. The biomaterial damage may be from non-uniform warming leading to overheating and/or thermomechanical stresses in the biomaterial. The biomaterial damage due to overheating may be from reaching a temperature at which excessive CPA toxicity occurs, reaching a temperature at which excessive ischemia occurs, or exceeding physiological temperatures to the point that thermal injury occurs.

In another aspect, the present description relates to a method of handling a biomaterial. The method can include cooling a biomaterial suspended in and/or perfused with a cryoprotective solution to a cryopreserved state to form a cryopreserved biological composition. The method can include applying resistive heating to rewarm the cryopreserved biological composition. The resistive heating can include discharging a tunable voltage pulse from an electrical generator to an electrical conductor, wherein the generator is electrically connected to the conductor and wherein the voltage pulse is tuned to generate heat sufficient to rewarm across the heat diffusion length of the biomaterial with minimal biomaterial damage. The cryopreserved biological composition includes a cryopreserved biomaterial in thermal contact with the conductor. The method may further include placing the biomaterial in thermal contact with the electrical conductor prior to or after cooling the biomaterial to the cryopreserved state. The heat diffusion length may be about the thickness of the biomaterial. The heat diffusion length may be about half the thickness of the biomaterial. The heat diffusion length may be between about 1 μm and about 10 mm. The voltage pulse discharged from the generator may be tuned to generate a minimum heating rate and a temperature change in the cryopreserved composition sufficient to rewarm the cryopreserved biomaterial with minimal biomaterial damage. The method may further comprise determining a voltage pulse width that generates rewarming across the heat diffusion length of the biomaterial. The voltage width may be tuned to achieve the minimum heating rate and the temperature change of the cryopreserved biomaterial. The voltage pulse width may be between about the heat diffusion time of the cryopreserved composition and about 10 times the heat diffusion time of the cryopreserved composition. The voltage pulse may have a pulse width of between about 0.1 μs and about 10 seconds. The cryoprotective solution may include cryoprotective agents selected from the group consisting of dimethyl sulfoxide, glycerol, propylene glycol, ethylene glycol, formamide, sucrose, glucose, glucose analogs, trehalose, raffinose, polyvinylpyrrolidone, and combinations thereof. The conductor may include stainless steel, titanium, platinum, aluminum, zinc, gold, silver, nickel, iron, chromium, molybdenum, silicon, carbon, ceramic, doped plastic and combinations thereof. The conductor can include a conductivity and geometry, wherein the conductivity and the geometry of the conductor are selected to generate the SAR to rewarm the biomaterial at a heating rate to minimize biomaterial damage. The geometry of the conductor may include a sheet, a mesh, a wire, a cylinder, a tube, a coil, a wound structure, a porous structure, a matrix structure, a channel and combinations thereof. The conductor configuration may be adapted to generate a sufficiently uniform heating rate across the heat diffusion length of the biomaterial to minimize biomaterial damage. The method may include an intermediate layer between the conductor and the biomaterial. The biomaterials are selected from the group consisting of adherent cells, droplets or thin films of cell suspensions, protein suspensions, embryos, oocytes, larvae, organisms, tissue slices, biopsies, isolated tissues, engineered tissues, cell clusters, organoids and the like. The voltage pulse form may be an exponentially decaying wave, sine wave, a rectangular wave, a triangular wave, a sawtooth wave or a square wave. The magnitude of the voltage pulse may be between about 1V and 10,000V. The method may be adapted for high throughput and/or larger size of the biomaterial. The biomaterial damage may be from ice crystallization causing damage to cellular and tissue structures. The biomaterial damage may be from non-uniform warming leading to overheating and/or thermomechanical stresses in the biomaterial. The biomaterial damage due to overheating may be from reaching a temperature at which excessive CPA toxicity occurs, reaching a temperature at which excessive ischemia occurs, or exceeding physiological temperatures to the point that thermal injury occurs.

In yet another aspect, the present description includes a system for rewarming a cryopreserved biological composition. The system includes an electrical generator electrically conducted to an electrical conductor. The electrical conductor is configured for thermal contact with a cryopreserved composition including a biomaterial. A discharge of a tunable voltage pulse from the electrical generator may generate heat sufficient to rewarm across the heat diffusion length of the cryopreserved composition with minimal biomaterial damage. The system may include generating a heating rate that is greater than the critical warming rate (CWR) of the cryopreserved biomaterial. The voltage pulse discharged from the generator may be tuned to generate a minimum heating rate and a temperature change in the cryopreserved composition sufficient to rewarm the cryopreserved biomaterial with minimal biomaterial damage. The voltage pulse width can generate rewarming across the heat diffusion length of the cryopreserved composition. The voltage width may be tuned to achieve the minimum heating rate and the temperature change of the cryopreserved biomaterial. The voltage pulse width may be between about a heat diffusion time of the cryopreserved composition and about 10 times the heat diffusion time of the cryopreserved composition. The voltage pulse may have a pulse width of between about 0.1 μs and about 10 seconds. The cryopreserved composition includes a biomaterial suspended in and/or perfused with a cryoprotective solution. The cryoprotective solution may include cryoprotective agents selected from the group consisting of dimethyl sulfoxide, glycerol, propylene glycol, ethylene glycol, formamide, sucrose, glucose, glucose analogs, trehalose, raffinose, polyvinylpyrrolidone, and combinations thereof. The conductor may include stainless steel, titanium, platinum, aluminum, zinc, gold, silver, nickel, iron, chromium, molybdenum, silicon, carbon, ceramic, doped plastic and combinations thereof. The conductor can include a conductivity and geometry, wherein the conductivity and the geometry of the conductor are selected to generate the SAR to rewarm the biomaterial at a heating rate to minimize biomaterial damage. The geometry of the conductor may include a sheet, a mesh, a wire, a cylinder, a tube, a coil, a wound structure, a porous structure, a matrix structure, a channel and combinations thereof. The conductor configuration may be adapted to generate a sufficiently uniform heating rate across the heat diffusion length to minimize biomaterial damage. The system may include an intermediate layer between the conductor and the cryopreserved composition. The biomaterials are selected from the group consisting of adherent cells, droplets or thin films of cell suspensions, protein suspensions, embryos, oocytes, larvae, organisms, tissue slices, biopsies, isolated tissues, engineered tissues, cell clusters, organoids and the like. The voltage pulse form may be an exponentially decaying wave, sine wave, a rectangular wave, a triangular wave, a sawtooth waves or a square wave. The magnitude of the voltage pulse may be between about 1V and 10,000V. The system may be adapted for high throughput and/or larger size of the biomaterial. The biomaterial damage may be from ice crystallization causing damage to cellular and tissue structures. The biomaterial damage may be from non-uniform warming leading to overheating and/or thermomechanical stresses in the biomaterial. The biomaterial damage due to overheating may be from reaching a temperature at which excessive CPA toxicity occurs, reaching a temperature at which excessive ischemia occurs, or exceeding physiological temperatures to the point that thermal injury occurs.

### BRIEF DESCRIPTION OF THE DRAWINGS

FIG. 1A is a schematic diagram of a rapid and scalable rewarming platform technique using Joule heating.

FIG. 1B is a plot showing the correlation between the biosystem thickness, pulse width and heat diffusion time.

FIG. 2A is a schematic of the R-C discharge circuit used to generate Joule heating by connecting the electrical conductor to a pulse generator. The voltage and pulse width can be adjusted. The capacitance is 4,000 μF. Ranges of the current, voltage and pulse width (pw) for the low voltage mode (LV, ≤500V) are listed in the figure.

FIG. 2B is a schematic of the electrical conductors, stainless steel (SS) sheet and SS mesh, used in the Examples described herein. The thickness of SS sheet is 12 μm. The wire diameter and aperture size of the SS mesh are 30 and 38 μm, respectively. The SS mesh allows easy removal of excess CPA solution.

FIG. 2C is a plot showing that the electrical conductivity (σ) of SS sheet and mesh can be calculated from the resistance by measuring the voltage and current. n=6. For FIG. 2C data is presented as mean±s.d.

FIG. 2D is a bar graph comparing various materials for generating rapid warming rates. The warming rate is inversely proportional to the product of material density (ρ), electrical conductivity (σ) and specific heat capacity (Cₚ). Details in equation 6 assuming same electrical current.

FIG. 2E is a plot of a voltage profile when different voltage pulse widths were used. The exponential decay of measured voltage matched with the calculated voltage of a RC discharge circuit.

FIG. 2F is a plot of the voltage profile when multiple voltage pulses were applied.

FIG. 2G are plots showing that the maximum energy delivered per pulse depends on the resistance and pulse width. For R≤1Ω, the max current of 500 A is used for calculation. For R≥1Ω, the max voltage of 500 V is used for calculation. The maximum current and voltage used are limitations of the pulse generator used in the Examples, but higher energy can be delivered at higher currents and voltages.

FIG. 2H is a plot of the temperature profile of a thin film resistor (150Ω) when subjected to a voltage pulse of 300 V and 5 ms. A thermocouple was attached to the electrically insulated surface of the resistor. The resistor was submerged in liquid nitrogen (LN₂) during the measurement. Sampling resolution is 1 ms.

FIG. 2I is a plot of the temperature change (ΔT) as a function of applied voltage. The same setup shown in panel FIG. 2H was used. n=6. For FIG. 2I data is presented as mean±s.d.

FIG. 3A are plots of simulated temperature profiles of the stainless steel (SS) sheet and cell monolayer under pulse widths (pw) ranging from 1 μs to 1 ms (shared y axis). Cells were grown and adherent to the SS sheet. The warming rates at the middle of SS sheet (point A), middle of cells (point B) and top of cells (point C) are displayed.

FIG. 3B is a plot of simulated temperature (in black) and measured viability (in red) of human dermal fibroblasts (HDFs) for 1 ms and 100 μs pulse width with different voltages. VS55 was used as the CPA (i.e., 55 wt % CPA). n=4. Data presented as mean±s.d.

FIG. 3C are images of merged Hoechst/Propidium Iodide (PI) images of HDF cells. Conditions include underheating, good warming, overheating (for Joule heating, as numbered in FIG. 3B) and positive control are shown. The pulse width is 1 ms. Scale bar is 500 μm.

FIG. 3D is a plot of estimated critical warming rate (CWR) plotted as a function of CPA concentration. Propylene glycol (PG, good glass forming tendency) and glycerol (poor glass forming tendency) were used to estimate the range of CWR (orange area) of different CPA concentrations. Concentrations of 13.75%, 27.5%, 41.25% and 55% CPA were tested. The measured convective warming rate (WR) is marked in the plot, along with the simulated WR for 1 ms, 100 μs, and 10 μs pulse Joule heating. The experimentally tested conditions are labeled as dots at the intersections of CPA concentrations and warming rates. Dots without filling that are located above the CWR (orange) area indicate minimal ice formation during warming. Dots with solid filling that are located below the CWR area indicate mostly ice formation. Dots with half filling that are located inside the CWR area indicate possible ice formation.

FIG. 3E is a plot showing the post thaw viability of HDF cells using different CPA concentrations and warming methods. Vitrification failure (i.e., ice formation) was noted for 13.27% and 27% CPA groups. n=4. Box and horizontal line represent standard deviation and mean respectively; whiskers represent max and min.

FIG. 3F are images of 13.75% CPA, merged Hoechst/PI images of HDF cells rewarmed by convective warming and 1 ms, 100 μs and 10 μs pulse Joule heating. Scale bar is 500 μm. One-way ANOVA and Tukey's post hoc were used for statistical analysis. ns, p>0.05, * p<0.05, ** p<0.01, *** p<0.001.

FIG. 4A are images of the Drosophila embryos on the stainless steel (SS) mesh after cooling. Embryos were loaded with different CPA concentrations. Embryos appeared transparent (i.e., underlying wires clearly visible) if vitrified. Scale bar is 500 μm.

FIG. 4B is a schematic of the geometry and dimensions of the SS mesh and embryos used in the heat transfer modeling. Two embryos with different contact patterns with the mesh were included. Points A, B, C represent the top, middle and bottom of the embryo. Point D represents the SS mesh in contact with the embryo. Point E represents the SS mesh outside the embryo.

FIG. 4C is a schematic of the warming rate at the top, middle and bottom of the embryo (Point A, B, C, respectively) for different Joule heating pulse widths (0.1 ms, 1 ms, 10 ms, respectively).

FIG. 4D are plots of the temperature profile at different locations (point A to E) for 1 ms Joule heating.

FIG. 4E are plots of the warming rate distribution at the middle plane of embryos.

FIG. 4F are images of embryos on the SS mesh acquired by a high-speed camera at 3000 frames per second (fps). At t=50 ms, embryos appeared hazy (i.e., ice formation), followed by clear appearance (i.e., melting of ice) at t=150 ms. The CPA was 27% EG+9% sorbitol. The voltage was 290 V. Scale bar is 500 μm.

FIG. 4G shows plots of normalized grayscale intensity of the embryos plotted from the high-speed camera videos. Ice (i.e., white color) showed a high intensity value. For recrystallization and/or devitrification, the intensity first increased (ice forming) then decreased (ice melting). Different CPA concentrations (labelled as EG concentration+sorbitol concentration) were tested. n=5. Data presented as mean±s.d.

FIG. 4H is a plot of the survival of embryos after exposure to different CPA concentrations. Hatch rate (red) represents the survival from embryos to larvae. Adult rate (blue) represents the survival from larvae to adults. n=4.

FIG. 4I is a plot of the temperature of the embryos after 1 ms Joule heating using different voltages.

FIG. 4J is a plot of the survival of embryos after 1 ms Joule heating using different voltages. The CPA was 27% EG+9% sorbitol. n=8.

FIG. 4K is a plot of the comparison of embryo survival using convective warming and Joule heating (290V, 1 ms) for different CPA concentrations. n=8. For FIGS. 4H, 4J and 4K box and horizontal line represent standard deviation and mean respectively; whiskers represent max and min. For FIGS. 4C, 4D, 4E and 4I modeling results were shown. Multivariate analysis of variance (MANOVA) and Tukey's post hoc were used for statistical analysis. ns, p>0.05; *** p≤0.001; **** p≤0.0001.

FIG. 5A is a schematic of the dimensions of the rat kidney slices used for the Joule heating. Kidney slices of 1.2 mm thickness and 6 mm in diameter were used. After CPA loading, the kidney slice was sandwiched using the SS mesh prior to plunge-cooling in liquid nitrogen, then rewarmed by connecting the SS mesh to a voltage source.

FIG. 5B is a plot of the warming rate distribution within the kidney slice. Point A represents the SS mesh; B, C and D represent the kidney slice. The detailed locations of point A, B, C, D were marked in the plot.

FIG. 5C are plots of the temperature profile at different locations (point A to D) for 100 ms Joule heating.

FIG. 5D is a plot of the warming rates at different locations within the kidney slice using 10 ms, 100 ms and 1000 ms Joule heating.

FIG. 5E are plots of predicted CPA concentration distribution within the kidney slice after loading with different CPA concentrations.

FIG. 5F is a plot of viability of kidney slices after exposure to different CPA concentrations. n=9.

FIG. 5G is a plot of post thaw viability of the kidney slices by Joule heating using different voltages. The CPA is 50% VS55. n=8.

FIG. 5H is a plot of comparison of post thaw viability of kidney slices using convective warming and Joule heating for different CPA concentrations. n=8.

FIG. 5I are images of histology (H & E staining) of kidney slices. Scale bar is 500 μm. Modeling results were shown in FIGS. 5B-5E. For FIGS. 5F-5H, the viability was measured by alamarBlue assay and normalized by the readings prior to treatment. Data presented as mean±s.d. One-way ANOVA and Tukey's post hoc were used for statistical analysis. ns, p>0.05, * p<0.05, ** p<0.01, *** p<0.001.

FIG. 6A is a plot showing the correlation between pulse width and biosystem heat diffusion length. To achieve rapid and sufficiently uniform rewarming (area in red), the pulse width (tₚ) can be selected based on heat diffusion length of the biosystems. When the pulse width is much smaller than the heat diffusion time (t_{d}), non-uniform warming occurs. For longer pulse width (i.e., >10 t_{d}), the achieved rewarming rate is slow. It is assumed that sufficiently uniform rewarming can be achieved using tₚ ranging from t_{d} to 10 t_{d}.

FIG. 6B is a plot showing the correlation between biosystem warming rate and biosystem heat diffusion length. Achievable warming rate inside the biosystems as a function of heat diffusion length is shown. The black and red lines represent the warming rates when tₚ=t_{d} and tₚ=10 t_{d}, respectively. The achieved warming rate decreases with increasing pulse width.

FIG. 6C is a plot showing the correlation between the CPA concentration and biosystem heat diffusion length. FIGS. 6A-6C holistically provide a means for selection of CPA concentration for optimal cryopreservation of different biosystems using external Joule heating source within the “rapid and uniform warming” region defined in FIG. 6A. The upper bound of CPA concentration (green line) is estimated using the longer pulse width (i.e., tₚ=10 t_{d}) and glycerol as the CPA (i.e., relatively high CWR). The lower bound of CPA concentration (blue line) is estimated using the shorter pulse width (i.e., tₚ=t_{d}) and PG as the CPA (i.e., relatively low CWR). The CPA toxicity is the major failure mode of cryopreservation in the upper corner region (i.e., orange color). Devitrification is the major failure mode of cryopreservation in the lower corner region (i.e., blue color).

FIGS. 7A-7D are plots of specific absorption rate (SAR) of the stainless steel (SS) sheet Joule heating for adherent cell cryopreservation. The SAR profiles of 1 μs (FIG. 7A), 10 μs (FIG. 7B), 100 μs (FIG. 7C) and 1 ms (FIG. 7D) voltage pulse were used to model the temperature profile shown in FIG. 2A. The resistance and voltage values are shown in the plot.

FIG. 8 is a plot of the measured convective cooling and warming rate of the stainless steel (SS) sheet for adherent cells cryopreservation. n=5.

FIGS. 9A-9C are plots of the simulated temperature and specific absorption rate (SAR) profiles of the stainless steel (SS) mesh Joule heating for Drosophila embryos cryopreservation. The SAR profiles (lower panel) of 10 ms (FIG. 9A), 1 ms (FIG. 9B), and 0.1 ms (FIG. 9C) voltage pulse were used to model the temperature profiles (upper panel). The resistance and voltage values were shown in the plot. Point E represents the SS mesh outside the embryo.

FIGS. 10A-10B are images of Drosophila embryos on the stainless steel (SS) mesh acquired by a high-speed camera at 3000 fps. For both 21% EG+9% sorbitol (FIG. 10A) and 27% EG+9% sorbitol (FIG. 2B), after 1 ms pulse at 290 V Joule heating (applied at t=~25 ms). In FIG. 10A, the embryos first appeared white (i.e., recrystallization/devitrification at t=68 ms), followed by the melting of ice at t=150 ms. In FIG. 10B., with 6% additional EG shows a lower degree of ice formation (i.e., less intense white and more transparent—i.e., vitrified embryos). The normalized grayscale intensity of the embryos was shown in FIG. 4G. Scale bar is 500 μm.

FIGS. 11A-11D are plots of survival of Drosophila embryos after 1 ms Joule heating using different voltages (230-330 V) and CPA concentrations. The tested CPAs include (FIG. 11A) 21% EG+9% sorbitol, (FIG. 11B) 27% EG+9% sorbitol, (FIG. 11C) 33% EG+9% sorbitol and (FIG. 11D) 39% EG+9% sorbitol. Hatch rate (red) represents the survival from embryos to larvae and adult rate (blue) represents the survival from larvae to adults. n=8. Box and horizontal line represent standard deviation and mean respectively; whiskers represent max and min.

FIGS. 12A-12C are plots of the simulated temperature and specific absorption rate (SAR) profiles of the stainless steel (SS) mesh Joule heating for kidney slice cryopreservation. The SAR profiles (lower panel) of 1000 ms (FIG. 12A), 100 ms (FIG. 12B), and 10 ms (FIG. 12C) voltage pulse were used to model the temperature profiles (upper panel). The resistance and voltage values are shown in the plot.

FIGS. 13A-13D are plots of the simulated temperature of the stainless steel (SS) mesh Joule heating for 100 ms pulse rewarming of kidney slices using different voltages. Voltages including (FIG. 13A) 100 V, (FIG. 13B) 200 V, (FIG. 13C) 300 V, and (FIG. 13D) 350 V were investigated. The final temperature after pulse heating is labeled in the plot.

FIGS. 14A-14C are plots of post thaw viability of the kidney slices by Joule heating using different voltages and 100 ms pulse duration. The tested CPAs include (FIG. 14A) 50% VS55, (FIG. 14B) 62.5% VS55 and (FIG. 14C) 75% VS55. The viability was measured by alamarBlue assay and normalized by the readings prior to treatment. Data presented as mean±s.d. n=8.

FIG. 15 shows the geometry and mesh used for heat transfer simulation of (a) adherent cells, (b) Drosophila embryos and (c) kidney slice in Joule heating.

### DETAILED DESCRIPTION OF ILLUSTRATIVE EMBODIMENTS

Various terms are defined herein. The definitions provided below are inclusive and not limiting, and the terms as used herein have a scope including at least the definitions provided below.

The terms “preferred” and “preferably”, “example” and “exemplary” refer to embodiments that may afford certain benefits, under certain circumstances. However, other embodiments may also be preferred or exemplary, under the same or other circumstances. Furthermore, the recitation of one or more preferred or exemplary embodiments does not imply that other embodiments are not useful, and is not intended to exclude other embodiments from the inventive scope of the present disclosure.

The singular forms of the terms “a”, “an”, and “the” as used herein include plural references unless the context clearly dictates otherwise. For example, the term “a tip” includes a plurality of tips.

Reference to “a” chemical compound refers to one or more molecules of the chemical compound, rather than being limited to a single molecule of the chemical compound. Furthermore, the one or more molecules may or may not be identical, so long as they fall under the category of the chemical compound.

The terms “at least one” and “one or more of” an element are used interchangeably, and have the same meaning that includes a single element and a plurality of the elements, and may also be represented by the suffix “(s)” at the end of the element.

The terms “about” and “substantially” are used herein with respect to measurable values and ranges due to expected variations known to those skilled in the art (e.g., limitations and variability in measurements).

The terms “and/or” means one or all of the listed elements or a combination of any two or more of the listed elements.

The terms “comprises,” “comprising,” and variations thereof are to be construed as open ended—i.e., additional elements or steps are optional and may or may not be present.

Unless otherwise specified, temperatures referred to herein are based on atmospheric pressure (i.e. one atmosphere).

The term “cryopreservation” as referred to herein relates to preservation of a biological sample/specimen at cryogenic temperatures. Cryopreservation includes cooling/freezing the biological sample below the freezing point of the sample in such a manner as to prevent significant damage due to ice crystallization and to a sufficiently low temperature in order to slow/arrest metabolic/chemical activity which can provide long term storage of biomaterials. Cryopreservation of a biological sample can also include warming the biological sample to recover the function/activity of the biological sample after storage at cryogenic temperatures.

The term “cryogenic” or “cryogenic temperature” as referred to herein relates to a temperature below sub-zero. Cryogenic temperatures can be in the range from −80° C. (112° F.) to absolute zero (−273° C. or −460° F.), but includes any effects below the freezing point of the sample/specimen.

The term “cryopreserved state” as referred to herein relates to a cryopreserved composition at a cryogenic temperature.

The term “cryogenic coolant” as referred to herein relates to a substance that is at a cryogenic temperature, e.g., liquid nitrogen, slush nitrogen.

The term “cryoprotective solution” or “CPA cocktail” or “cryoprotective composition” as used herein relates to a solution that includes one or more cryoprotective agent(s) (CPA(s)). Cryoprotective solution may be referred to as “CPA solution” or “CPA”. “Cryoprotective solution”, “CPA solution”, “CPA cocktail”, “cryoprotective composition” and “CPA” are used interchangeably herein.

The term “suspended in” as used herein relates to a biomaterial that is surrounded by cryoprotective solution prior to the cooling step. A biomaterial that is suspended in cryoprotective solution prior to the cooling step will have already been loaded with cryoprotective solution as is known by a person skilled in the art, so as to permit successful cryopreservation with minimal biomaterial damage. Loading of cryoprotective solution may include, for example, diffusion and/or perfusion loading.

The term “perfused with” as used herein relates to loading a biomaterial with cryoprotective solution by passing the cryoprotective solution through vessels or other natural channels within the biomaterial as is known by a person skilled in the art.

The term “Joule heating” or “resistive heating” or “ohmic heating” as referred to herein relates to heat generation by passing an electric current through a conductor. Joule heating, resistive heating and ohmic heating can be used interchangeably herein.

The term “electrical conductor” or “conductor” as referred to herein relates to a material that allows flow of charge such as electric current in one or more directions with the application of voltage.

The term “electrical generator” or “generator” as referred to herein relates to a device that can discharge sufficient voltage/current and provide tunable voltage pulses. The generator can provide a tunable voltage pulse with respect to magnitude of voltage or magnitude of the pulse, duration of the pulse or pulse width, and pulse waveform. “Magnitude of the pulse” and “magnitude of voltage” can be used interchangeably herein.

The term “thermal contact” as referred to herein relates to contact between two elements, e.g., biospecimen and conductor, in a manner that allows for transfer of thermal energy from one element to the other through conduction. The biospecimen and the conductor may or may not be in direct contact. There may be one or more intermediate layer(s) between the biospecimen and the conductor. The intermediate layer(s) may be, for example, a film, a liquid coating, a powder coating and combinations thereof. Thermal contact can occur as long as the proximity between the biospecimen and the conductor allows for the transfer of thermal energy commensurate with the described effects.

The term “voltage pulse” as referred to herein relates to a pulse wave of voltage discharged by an electrical generator. Characteristics of a voltage pulse include pulse duration or width, voltage (or pulse) magnitude and pulse waveform. The magnitude of the pulse wave (or applied voltage), duration of the pulse wave and the pulse waveform can vary for a rewarming application and/or during a rewarming process. The magnitude of the pulse wave can be constant during the duration of the pulse wave or variable during the duration of the pulse wave.

The term “pulse width” or “pulse duration” or “voltage pulse width” as referred to herein relates to the elapsed time between the leading and trailing edges of a pulse of energy or voltage. Pulse width and pulse duration and voltage pulse width are used interchangeably herein.

The term “temperature change” as used herein relates to increasing the temperature of a cryopreserved biomaterial at a cryogenic temperature to a selected temperature above the melting point of the cryopreserved biomaterial. Typically, the cryopreserved biomaterial will be rewarmed from a cryogenic temperature to a temperature above the melt temperature of the cryopreserved biomaterial, but below a temperature at which significant CPA toxicity or thermal damage is expected. Certain biological specimens can also experience chilling injury at lower temperatures and the selected final temperature may also be above a temperature to avoid chilling injury.

The term “cryomesh” as referred to herein relates to a cryoresistant material/tool that can handle a biological sample. The cryomesh can, for example, retain a biological sample on the filaments of the mesh while enabling the removal of any cryoprotective solution surrounding the biological sample.

The term “heat diffusion length” as referred to herein relates to the distance that heat conducts through a biomaterial to ensure the entire biomaterial has been rewarmed from the cryopreserved state. The heat diffusion length can be, for example, the thickness of the biomaterial. The heat diffusion length can refer to, for example, the thickness of the biomaterial, but can depend on the geometry of the biomaterial (e.g., planar, cylindrical, spherical, etc. geometries) and the geometry of rewarming (e.g., rewarming from multiple sides).

The term “heat diffusion time” is the characteristic time that it takes heat to diffuse across a distance (e.g., the heat diffusion length). It can be calculated for example based on a planar geometry as: τ_{d}=(h²/2α), where h is the thickness of the biomaterial and α is the thermal diffusivity of the cryopreserved biomaterial. Example calculations throughout assume a thermal diffusivity of 10⁻⁶ m²/s, which is that of 2M glycerol laden tissue at cryogenic temperatures (Choi and Bischof. Cryobiology 2010), which should be representative of behavior across a range of similar systems. However, direct calculation of the heat diffusion time can be made for any biomaterial system of interest.

The term “sufficiently uniform warming” as referred to herein relates to heating which maintains the temperature difference within a biomaterial sufficiently low as to prevent damage to the biomaterial from overheating (e.g., thermal damage or increased toxicity at higher temperatures) and/or thermomechanical stresses in the specimen (e.g., cracking).

The term “vitrification” as referred to herein relates to a biological sample that has attained a glassy, amorphous structure without significant ice crystallization when cryopreserved. Vitrified biospecimen can be stored at cryogenic temperatures (e.g., <−150° C.) in an ice-free glassy state for indefinite periods of time.

The term “minimal biomaterial damage” as referred to herein relates to rewarming a cryopreserved biomaterial to produce a viable biological sample. The biomaterial may sustain none or little damage during the rewarming process, so as to maintain its intended function after cryopreservation.

The term “devitrification” as referred to herein relates to a vitrified biological sample that has experienced significant ice crystal growth and some biomaterial damage and may not produce a viable biological sample upon warming to room or physiological temperature.

The term “crystallized” sample as referred to herein relates to a biological sample that has experienced significant ice crystal growth and may not produce a viable biological sample upon warming to room or physiological temperature. Crystallized samples may also be referred to herein as unvitrified samples, non-vitrified samples, or devitrified samples. These terms are used interchangeably herein.

The term “vitrified and rewarmed” or “VR” as referred to herein relates to a vitrified and rewarmed biological sample. The VR sample may be used in biological/therapeutic application. Vitrification and rewarming may be performed in a variety of ways.

The term “high-throughput” as referred to herein relates to methods to rapidly process a large number of samples in a short amount of time.

The term “scalable” as referred to herein relates to methods that can be adapted to increase throughput and/or adapted to larger and/or smaller biomaterials.

The term “platform” or “system” as referred to herein relates to the use of a technology that can be adapted for a variety of applications based on the applied principles of the invention.

The term “Joule heating platform” as referred to herein relates to the use of Joule or resistive heating in rewarming methods and adapted for rewarming of the cryopreserved biospecimen of interest.

The term “biological specimens”, “biospecimen”, “biosystem”, “biological samples”, “biological material” and/or “biomaterials” are used interchangeably herein and as referred to herein relate to cells, cell clusters, adherent cells, germplasm, cell aggregates, spheroids, organoids, cell suspension, droplets or thin films of cell suspensions, protein suspensions, embryos, islets, oocytes, larvae, organisms, tissues, tissue slices, isolated tissues, engineered tissues, biopsies, organoids, and the like. The germplasm can be from a variety of species including, for example, coral germplasm, mammalian germplasm, invertebrate germplasm and the like. The biological samples can be unicellular organisms such as bacteria, protozoa and the like. The oocytes can be, for example, from invertebrates such as Drosophila, mosquito and others, and vertebrates such as fish, amphibians, mammals, humans and others. The tissues can be precision cut tissue slices, biopsies, cell clusters, organoids and the like. The biological samples can be related to commercially relevant or endangered species (i.e. regenerative medicine, transplant medicine, biomedical research, therapeutic discovery and screening, agriculture, aquaculture and biodiversity).

The term “organoid” as referred to herein relates to a 3D multicellular in vitro tissue construct that mimics its corresponding in vivo organ such that it can be used to study or provide functional aspects of that organ.

The term “critical cooling rate” or “CCR” as referred to herein relates to the rate of temperature change needed to avoid significant ice crystal formation during cooling.

The term “critical warming rate” or “CWR” as referred to herein relates to the speed of temperature rise needed to avoid significant ice crystal formation during rewarming.

The term “micrometer” sample as referred to herein relates to a biological sample that is equal to more than about a micrometer.

The term “sub-millimeter” sample as referred to herein relates to a biological sample that is equal to or less than about a millimeter.

The term “millimeter” sample as referred to herein relates to a biological sample that is equal to or more than about a millimeter.

Biological samples can include other components to aid in the cryopreservation process, e.g., cryopreserving agent, buffer or other media that are present when the biological sample is prepared, transferred and/or cryopreserved. The size of the biological sample may be characterized by the thickness of the biological sample or specimen.

The present description includes methods and systems for cryopreservation of biological materials. The description can include methods and system for rewarming of the cryopreserved biological materials. Methods for cryopreservation and rewarming of micrometer, sub-millimeter and/or millimeter scale biological materials are disclosed. The methods can be rapid and scalable (both in throughput and biosystem size) rewarming techniques.

The present description includes an innovative Joule heating platform and method for rewarming a variety of cryopreserved biomaterials. The method can include rapidly rewarming a cryopreserved biological composition in thermal contact with an electrical conductor. The method can include providing a voltage pulse from an electrically connected generator to the electrical conductor. The Joule heating method may be controlled by the applied voltage and pulse duration, and may convert almost 100% of the input electrical energy into thermal energy.

The present disclosure can provide methods for rewarming cryopreserved biological compositions of a broad spectrum of cellular, organismal and tissue based biomaterials using low CPA concentrations in a scalable manner. In various embodiments, the Joule heating platform methods disclosed herein can greatly improve the viability and function of the biological specimen compared to conventional convective warming methods.

In some exemplary embodiments, the method can include cryopreservation of three biosystems with thicknesses across three orders of magnitude, including adherent cells (~4 μm), Drosophila embryos (~50 μm) and rat kidney slices (~1.2 mm) using low CPA concentrations. In various embodiments, warming rates from 9×10⁴ to 6×10⁸° C./min can be achieved, a range exceeding any current rapid warming method for biosystems in these size ranges. See Table 1 below. The methods described herein can also include cooling a biological composition and/or rewarming a cryopreserved biological composition using Joule heating. The biological compositions can include biomaterial/biospecimen in a cryoprotective solution.

TABLE 1
Summary of novel rewarming methods *

| | | SAR | | | | Warming | |
|---|---|---|---|---|---|---|---|
| Warming | | reported | Heating | | Biomaterials | rate | |
| method | Mechanism | (W/m³) | volume/area | | tested & thickness | (° C./min) | Ref. |
| RF | RF heating | 2.5 × 10⁶ | 1-80 | ml | Porcine arteries | >300 | (Manucherhrbadi, |
| nanowarming | of IONP | | | | (~0.7 mm) | | Sci. Transl. |
| | | | | | Porcine heart valve | | Med., 2017; |
| | | | | | (~1.4 mm) | | Chiu-Lam, |
| | | | | | | | Science Adv., |
| | | | | | | | 2021; Gao, Adv. |
| | | | | | | | Science, 2020) |
| Metal form | RF heating | 3.5-6.5 × 10⁸ | 1.3 cm × 2.8 cm | | Porcine arteries | 1.3-2 × 10³ | (Han, Adv. |
| | of Al foil | | | | (1-2 mm) | | Healthcare Mat., |
| | | | | | | | 2020) |
| Focused IR | IR | NA | 200 | ul | Adherent cells | 2-30 × 10³ | (Bissoyi, |

| | | | | | | |
|---|---|---|---|---|---|---|
| light | absorption | | | (2~4 mm) | | Cryobiology, |
| | of water | | | | | 2021) |
| Laser | Laser | 4.4 × 10¹¹ | <10 ul | Coral larvae | 7 × 10⁴-1.2 × 10⁷ | (Cirino, Sci. |
| nanowarming | plasmonic | | droplet | (~100 μm) | | Reports, 2019; |
| | heating | | | Cells in suspension | | Zhan, Adv. |
| | | | | (~15 μm) | | Science, 2021; |
| | | | | | | Khosla, Adv. |
| | | | | | | Biosystems, |
| | | | | | | 2018) |
| Joule heating | Electrical | 3-600 × 10¹¹ | 15 cm × 1 cm | Adherent cells | 5 × 10⁴-6.8 × 10⁸ | This application |
| | current | | | (4 μm) | | |
| | heating | | | Drosophila | | |
| | | | | embryo | | |
| | | | | (50 μm) | | |
| | | | | Kidney slices | | |
| | | | | (1.2 mm) | | |

* Traditional convective warming using water bath is not listed here.
RF: radio frequency; IONP: iron oxide nanoparticle; Al: aluminum; IR: infrared radiation; SAR: specific absorption rate.

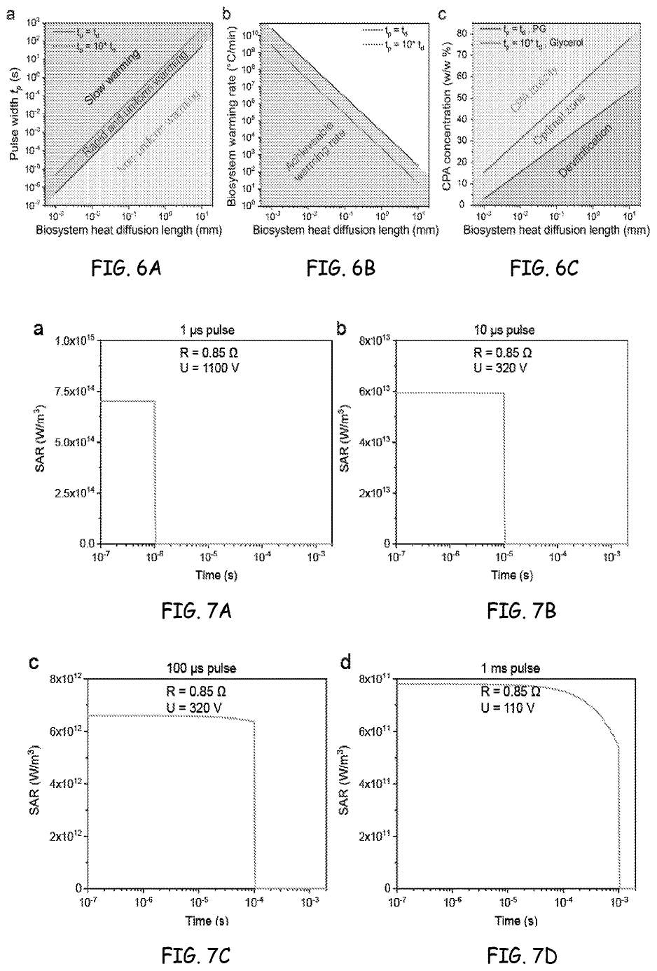

The amount of energy and power delivered by the Joule heating method can be adjustable enabling a wide range of warming rates in various Joule heating platform techniques. Precision voltage pulses can be readily generated with a pulse width duration of microseconds to minutes or longer. The magnitude of the voltage and the pulse width of the voltage pulse can be tailored to achieve rapid warming in broad ranges of biosystems across multiple scales. In some embodiments, a Joule heating platform can attain the selected rewarming rate at lower CPA loading for reduced toxicity and the possibility to rescue biological samples with partial ice formation. As shown in Table 1 and FIGS. 6A-6C, the Joule heating method can provide a wide range of warming rates. Advantageously, due to the intrinsic heating mechanism, the Joule heating technique can provide close to 100% input energy conversion to heat. Energy loss may occur in the resistance of the circuit (e.g., <0.1Ω), which is usually a small portion of the energy delivered.

In some embodiments, Joule heating can also generate higher specific absorption rate (SAR), e.g., up to about ~6×10¹³ W/m³, as compared to other heating methods (See Table 1). Advantageously, the heat source (i.e., electrical conductor) can be reusable, economical and available in large scale. In various embodiments, the conductors used to deliver the energy can be designed or configured to match the specific requirements of a given biological system. The material and/or the geometry of the conductors can be varied to match the specific requirements of a given biological system. In various embodiments, the material and/or the geometry of the conductors can be varied to match the requirements of a broader range of biological systems and the voltage pulse delivered by the generator can be varied to match the requirements of a given biological system. The Joule heating technique can be scaled for rewarming large quantities of biomaterials, for example, in large batches or a high-throughput manner. In some embodiments, the Joule heating platform can be configured, for example, into channel or matrix constructs to rewarm the biological system from multiple directions and in bulk volumes.

A variety of biological samples can be cryopreserved and rewarmed according to the methods described herein. In some embodiments, biological samples can be samples from terrestrial and/or aquatic organisms, such as embryos, larvae, whole organism, and the like. In various embodiments, biological samples can be, for example, cell clusters, spheroids, organoids, islets, hepatocyte clusters, 3-D cell clusters, and the like. In various embodiments, the biological material can be isolated and cultured from tissues and placed in a selected and/or a suitable media or buffer. In one embodiment, the biologic material can be cells or cell clusters adhered to the conductor or suspended in a solution. In one embodiment, the biological material may be in, for example, a buffer for maintaining the biological material prior to cryopreservation. In one embodiment, the biological material may be at a stage, e.g., a fully differentiated state, selected for cryopreservation.

In various embodiments, the biomaterial may be a droplet, layer, or channel of cellular suspension. In various embodiments, the biomaterial can be, for example, a tissue or a portion thereof that is perfused with or suspended in a cryoprotective solution as described herein. In various embodiments, the biomaterials can be, for example, embryos, tissues or tissue slices perfused with and/or suspended in a cryoprotective solution as described herein. In various embodiments, the biomaterials can be, for example, cells, cellular clusters, spheroids, organoids, embryos and the like suspended in a cryoprotective solution as described herein. The perfused and/or suspended biomaterial may be vitrified/cryopreserved and/or rewarmed when desired.

In various embodiments, the present description can include a system or platform for rewarming a cryopreserved biological composition. The platform or system can include a generator that is electrically connected to a conductor that can form a Resistor/Capacitor (R-C) discharge circuit. The generator can discharge electrical energy through voltage pulses of various magnitudes and characteristics as described below. The discharged voltage pulse is delivered through an electrical connection, e.g., through a cable, to the conductor and applied across the conductor to generate current that can heat the electrical conductor. The generator can be tunable in order to control the discharge of a voltage pulse with the selected characteristics.

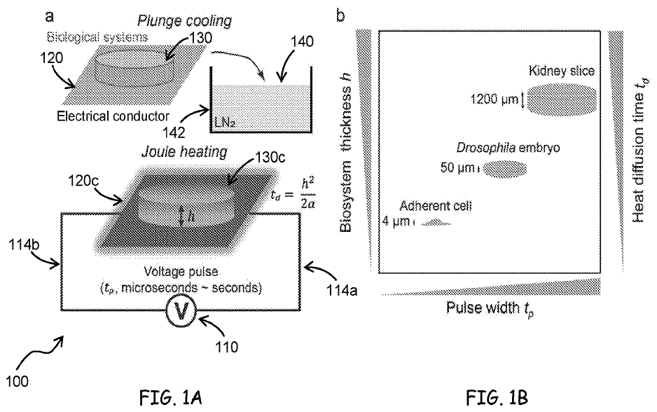

FIG. 1A is one exemplary embodiment of a schematic diagram of a system for Joule heating. System 100 includes generator 110 electrically connected to conductor 120c through cables 114a and 114b. Conductor 120c and biological system 130c are at a cryogenic temperature prior to discharge of a voltage pulse from generator 110. In one embodiment, biological composition 100 is placed on conductor 120 prior to plunge cooling in cryogenic coolant 140 in cryogenic container 142. In one embodiment, biological composition 100 may be plunge cooled prior to placement on conductor 120. A variety of configurations are possible for the system for Joule heating and all are within the scope of this description.

In one embodiment as shown in FIG. 1A, biological system 130 is loaded with cryoprotectant agent (CPA) solution and placed in thermal contact with electrical conductor 120. After removing the surrounding excess CPA solution, biological systems 130 and electrical conductor 120 were plunged together into liquid nitrogen (LN₂) 140 for cooling. For rewarming, electrical conductor 130c is connected to voltage pulse generator 110 to generate heat via Joule heating. Biomaterial 130c is rewarmed via thermal conduction from the electrical conductor.

FIG. 1B is a schematic of the three types of biosystems with different thicknesses that were used as the model systems for the methods described herein and include adherent cells (4 μm), Drosophila embryos (50 μm) and kidney slices (1.2 mm) as shown in the plot (drawings not to scale). The heat diffusion time (t_{d}, right y axis) across the biosystems is positively correlated with the biosystem thickness (h).

The present description can include a method for rewarming a biological composition in a cryopreserved state. In some embodiments, the method can include applying resistive heating of an electrical conductor and transferal of the heat from the conductor to a cryopreserved biomaterial in thermal contact with the conductor. In various embodiments, the electrical conductor is in thermal contact with a cryopreserved biological composition during rewarming of a cryopreserved biomaterial. The electrical conductor can be electrically connected to a generator described herein. A voltage pulse discharged from the generator can heat the electrical conductor. The direct ohmic or resistive heating of the conductor in the methods described herein can achieve ultrahigh warming rates under a voltage pulse. The method can also include using tunable voltage pulses to obtain the selected warming rates for a cryopreserved biospecimen. In one embodiment, the magnitude of the voltage pulse is tuned to a selected amount. In one embodiment, the duration of the voltage pulse is tuned to a selected pulse width. In one embodiment, the waveform of the voltage pulse is tuned to a selected shape. Without being bound by any theory, it is thought that because of the direct conversion of electrons to thermal energy, this method yields the fastest possible rates of rewarming at the boundary of a resistive material. Advantageously, the format of the Joule heating methods can achieve rapid and scalable rewarming of cryopreserved biosystems at different scales. In some embodiments, the biomaterial may be in a low CPA concentrations environment.

In various embodiments described herein, to address the potential for biomaterial damage from electrical current flowing through the biosystem, it should be highlighted that the electrical conductivity of cells and tissues are commonly on the order of 1 S/m or lower (J. Peters 2001, Haemmerich, Staelin et al. 2003, Gabriel, Peyman et al. 2009). In various embodiments, the electrical conductivity of the electrical conductor will be several orders of magnitude higher than that of the biosystem and so a majority of the electrical current will flow through the conductor. In various embodiments, a thin, thermally conductive, electrically insulating layer may be coated on the electrical conductor to minimize electrical current flow through the biosystem.

The methods described herein consider whether the Joule heating is uniform within the electrical conductor during a short voltage pulse. For example, as alternating electric current (AC) passes through a conductor, the rapid change of electrical field could lead to an additional internal induction field, resulting in higher current densities toward the surface of the conductor. In various embodiments described herein, the electrical current flows mainly within the “skin depth” from the outer surface of the conductor, increasing the resistance and causing inhomogeneous Joule heating within the conductor (e.g., higher heating at the outer surface of the conductor) (Han, Sharma et al. 2020).

The skin depth (δ) can be calculated as:

$$\partial = \sqrt{\frac{1}{π f σ μ}} \qquad ( 1 )$$

Where f is the frequency, σ is the electrical conductivity of the conductor, and μ is the magnetic permeability of the conductor. In one exemplary embodiment, where the conductor is stainless steel (SS) with f is ~1/pulse width, σ is 3.57×10⁵ S/m, and μ is 1.26×10⁻⁶ H/m, the skin depth is estimated to be 2.7 mm for a 10 μs pulse, which is two orders of magnitude larger than e.g. the thickness of a 12 μm stainless steel sheet, indicating uniform heat generation will occur within the SS sheet. In various other embodiments where the heat generation within the electrical conductor may be inhomogeneous, thermal conduction within the electrical conductor will dominate heat conduction into the biological system, so the heater will thermally equilibrate on the timescales of heat conduction within the biological system.

A variety of generators may be used in a Joule heating system and all are within the scope of this description. In various embodiments, the generator can deliver the selected voltage pulse for the biomaterial of interest. The generator can deliver a voltage pulse of the selected magnitude, duration and waveform. In one exemplary embodiment, a commercial pulse generator (ECM® 830, BTX® Harvard Apparatus, Holliston, MA) was used but it will be understood that there are a variety of other generators that can be used to practice the methods described herein.

In some embodiments, the generator may be used in a low voltage mode. Other voltage modes may also be used. In some embodiments, the voltage of the RC circuit can be less than about 10,000V, or less than about 5,000V, or less than about 1000V, or less than about 500V, or less than about 400V, or less than about 300V, or less than about 200V, or less than about 100V.

In one exemplary embodiment, a generator can include a capacitor with a capacitance of about 4000 μF and that can discharge a voltage pulse with pulse width ranging from about 10 μs to about 10 s and voltage from about 5V to about 3000V.

A variety of electrical conductors may be used in the Joule heating platform methods described herein and all are within the scope of this description. In various embodiments, the electrical conductors can include a single type of material. Alternatively, the electrical conductors can include a combination of materials. The electrical conductors can be, for example, stainless steel, titanium, platinum, aluminum, zinc, gold, silver, nickel, iron, chromium, molybdenum, silicon, carbon, ceramic, doped plastic and the like. The electrical conductors can include, for example, a combination of stainless steel, titanium, platinum, aluminum, zinc, gold, silver, nickel, iron, chromium, molybdenum, silicon, carbon, ceramic, doped plastic and the like.

Selection of the materials for the electrical conductor can depend on a variety of factors and can include, for example, the electrical conductivity, the material density, the heat capacity, the thermal conductivity, the price, the biocompatibility, the surface adhesion, the surface hydrophilicity/hydrophobicity and the like. In one embodiment, the conductor can be selected based on the electrical conductivity of the material. The resistive heating of a material can be inversely proportional to its conductance. A lower conductivity material can generate more heat at a fixed current. However, the resistive heating can also depend on the square of the current passing through the conductor and so highly conductive materials can also generate substantial heating at higher voltages (i.e., higher currents) or smaller cross-sectional area (i.e., higher resistance). In various embodiments, the material conductivity for the conductor can be, for example, between about 10⁵ and about 10⁸ Siemens/meter (S/m). In some embodiments, the material conductivity for the conductor can be, for example, between about 10⁶ and about 10⁷ Siemens/meter (S/m).

In some embodiments, the material density and heat capacity are both inversely proportional to the heating rate generated by the material and thus, conductor material may also be selected based on lower density and heat capacity. In some embodiments, the electrical conductor's thermal conductivity, in addition to material density and heat capacity, may influence the rate at which heat is transferred from the electrical conductor to the biomaterial and so a material with higher thermal conductivity may be desired. In some embodiments, considerations such as material cost, biocompatibility, and surface adhesion, i.e., how well the biospecimen remains in contact with the conductor, may be considered dependent on the specific cryopreservation application.

In some embodiments, the specific absorption rate (SAR) of the conductor can be correlated to the target specific warming rate of the biomaterial. To achieve rapid and sufficiently uniform rewarming, the conductor SAR can be selected based on the target warming rate of the biomaterial. Without being bound by any theory, the warming rate of the conductor is determined from the conductor SAR and the specific heat capacity and density of the conductor (i.e., conductor warming rate equals the conductor SAR/(conductor specific heating capacity×conductor density)). The product of the conductor specific heat capacity and density is not expected to vary significantly with material choice, so in one embodiment, stainless steel may be selected to represent expected behavior ((7750 kg/m³×470 J/kg-° C.) 4×10⁶ J/m³-° C.). The conductor warming rate that is much greater than the target biomaterial warming rate can lead to non-uniform warming (potential overheating and/or thermomechanical stresses in the specimen). For slower conductor warming rates, the achieved biomaterial warming rate may be too slow and lead to damage from ice crystallization during warming. In some embodiments, sufficiently rapid and uniform rewarming can be achieved when the conductor SAR is between about (selected biomaterial warming rate×(4×10⁶ J/m³-° C.)) and about (10×selected biomaterial warming rate)×(4×10⁶ J/m³-° C.)). Conductor SAR outside of this range may also be used and are within the scope of this description.

In some embodiments, the SAR range of the conductor can be from about 1×10⁵ to 1×10¹⁶ W/m³. The target SAR can depend on the properties of the conductor material, the thickness of the biomaterial, and the selected CPA concentration in the cryopreserved biological composition. FIGS. 6A-6C illustrates the dependency of these parameters on the heat diffusion length. In one exemplary embodiment, selection of a biomaterial for cryopreservation can determine the heat diffusion length. The heat diffusion length can determine the selected Joule heating warming rate for the biosystem. The selection of CPA can be determined by the selected warming rates and toxicity with the selected biomaterial. The conductor material and geometry can be selected to produce the required SAR and thermal contact for the biomaterial. The voltage pulse width can be selected based on the biomaterial heat diffusion length, so as to provide rapid and uniform warming. The voltage pulse width can additionally be selected based on the selected temperature change in the biomaterial and the selected warming rate.

The electrical conductors that can be used in the methods described herein can include a variety of shapes, configurations, sizes, thicknesses and all are within the scope of this description. In some embodiments, the electrical conductor can include a sheet or other flat configuration. The sheet may include a rim or other structure to retain the biological composition on the sheet. The thickness of the sheet can vary and the selection of the thickness of the sheet can be dependent on the cryopreserved biological composition and selected electrical resistance of the electrical conductor. In various embodiments, the thickness of the conductor sheet can be, for example, at least about 0.1 μm, or at least about 1 μm or at least 10 μm, or at least 20 μm, or at least 50 μm, or at least 100 μm, or at least 200 μm, or at least 500 μm, or at least 1000 μm. Thickness of the electrical conductor sheet outside of this range are also within the scope of this description. In one embodiment, the thickness of the conductor sheet can be between about 5 μm and about 50 μm, or between about 5 μm and about 20 μm.

Electrical conductors can be configured to have various geometries and all are within the scope of this description. In some embodiments, the electrical conductor can include a mesh structure, a porous structure and/or a wound structure. The wound structure can have a wire element that is wound or coiled in a regular or irregular manner. The porosity of the porous structure can be regular or irregular. The biomaterial to be rewarmed can be placed in thermal contact with the conducting structure. The mesh, porous or wound structure can include any of the materials disclosed above. The diameter of the wires to generate the wound structure and/or the mesh can vary and all are within the scope of this description. The diameter of the wires, size of pores and the aperture size can be, for example, dependent on the size of the biomaterial. In some embodiments, the diameters of the wires can be, for example, at least about 1 μm, or at least 10 μm, or at least 20 μm, or at least 50 μm, or at least 100 μm, or at least 200 μm, or at least 500 μm, or at least 1000 μm. Diameters of the wire outside of this range are also within the scope of this description.

The pore or aperture sizes in the mesh can vary and all are within the scope of this description. The aperture size in the mesh or wound structure can be dependent on the size of the biomaterials. The aperture size of the mesh can be small enough such that the biomaterial is retained on the mesh and does not pass through the aperture on the mesh. The aperture size in the porous or wound structure can vary and be dependent of the size of the biomaterials. The aperture size can be large enough such that the biomaterial can be positioned within the pore or aperture in the structure. In some embodiments, the aperture size of the mesh can be, for example, at least about 1 μm, or at least 10 μm, or at least 20 μm, or at least 50 μm, or at least 100 μm. Aperture sizes of the mesh outside of this range are also within the scope of this description. In some embodiments, the size of the pore or aperture can be, for example, at least about 1 μm, or at least 10 μm, or at least 20 μm, or at least 50 μm, or at least 100 μm, or at least 200 μm, or at least 500 μm, or at least 1000 μm. Pore or aperture sizes outside of this range are also within the scope of this description.

The geometry of the electrical conductor may include other configurations such as cylinders, boxes, sandwich plates, channels, radial configurations, and the like. In some embodiments, the electrical conductors may be cylinders or channels and the cryopreserved biological specimen may be within the cylinder, tube or channel during rewarming. In some embodiments, the cryopreserved biological specimen may be rewarmed from multiple directions, for example, between two sheets, in a channel, in a tube, in a recess and the like. Various materials in different form factor design can extend the Joule heating to achieve controlled spatial warming rates for cryopreservation of planar or radial systems such as cartilage, blood vessels and more complex tissues. In these embodiments, the ability to warm from multiple directions, or from a boundary inward (i.e. radial system) may allow achieving higher warming rates as needed to avoid crystallization failure in lower CPA loaded tissue (i.e. at points farther away from the boundary). In various embodiments, the conductor can be a porous material or structure where biomaterials are embedded within the porous structures for reduced heat diffusion length.

In one embodiment, the electrical conductor can be a stainless steel sheet. The thickness of the stainless steel sheet can be between about 5 μm and about 20 μm, or between about 5 μm and about 15 μm. Thickness of stainless steel sheets outside of this range are also within the scope of this description.

In one embodiment, the electrical conductor can be a stainless steel mesh. The diameter of the wires in the stainless steel mesh can be between about 20 μm and about 50 μm, or between about 25 μm and about 35 μm. The aperture size of the stainless steel mesh can be between about 20 μm and about 60 μm, or between about 25 μm and about 45 μm. The diameter of the wire and the size of the aperture of the stainless steel mesh outside of this range are also within the scope of this description.

In various embodiments, the methods described herein can include rewarming the cryopreserved biological composition when the cryopreserved composition is in thermal contact with the conductor. In one embodiment, the biomaterial is placed in direct contact with the conductor without any intermediate layer(s) between the conductor and the biological composition. In one embodiment, there may be one or more thin intermediate layers between the conductor and the biological composition. In some embodiments, the intermediate layer can enhance the cooling and/or the warming rate, can provide electrical insulation, can increase biocompatibility, and/or increase the hydrophilicity/hydrophobicity and/or increase surface adhesion of the electrical conductor. In various embodiments, the one or more intermediate layers can be films and/or coatings. The intermediate layer can be a liquid coating and/or a powder coating. In various embodiments, thin film coating materials may include, for example, metals, polymers, plastics, dielectrics, ceramics and the like. In some embodiments, the layer or coating may be a film. In some embodiments, the layer or coating may be a liquid or powder that is spread on the conductor using any known method of coating. The biomaterial may be placed on the conductor with an intermediate layer prior to cooling to a cryogenic temperature. The biomaterial may be placed on the conductor with an intermediate layer after the biomaterial is cooled to a cryogenic temperature.

In various embodiments, the rewarming method can include connecting a voltage pulse generator to an electrical conductor in thermal contact with a cryopreserved biological composition. The method can include rewarming a cryopreserved biological composition by applying Joule heating. The method can include discharging a voltage pulse from the generator to the conductor to deliver energy and generate high heating rates by Joule heating. High heating rates as used herein relates to heating rates that generate uniform heating of the biomaterial during rewarming from a cryopreserved state. The characteristics or parameters of the discharged pulse can be optimized for a target biomaterial in the cryopreserved composition to achieve uniform rewarming with high heating rates.

A variety of waveforms of the voltage pulses can be used and all are within the scope of this description. In some embodiments, an exponentially decaying voltage pulse can be used. In some embodiments, a square wave pulse form may be used. In some embodiments, the pulse forms can be sine waves, rectangular waves, triangular waves and/or sawtooth waves. Other forms of voltage pulses for controlled temporal warming rates may also be used and all are within the scope of this description.

In various embodiments, the voltage pulse can be advantageously tunable to a selected pulse width. The selected pulse width can vary and may be dependent on the conductor, the biomaterial, the selected heating rate, the specimen starting temperature, the selected end temperature of the specimen and the like.

In some embodiments, the pulse width can be adapted to generate heat sufficient to rewarm across the heat diffusion length of the biomaterial with minimal biomaterial damage. To achieve rapid and uniform rewarming, the pulse width (tₚ) can be selected based on heat diffusion length of the target biomaterial. Without being bound by any theory, the pulse width that is much smaller than heat diffusion time (t_{d}) to traverse the thickness of the biomaterial to be rewarmed can lead to non-uniform warming resulting in potential overheating and/or thermomechanical stresses in the specimen. For longer pulse width (i.e., >10 t_{d}), the achieved rewarming rate may be too slow and may lead to damage from ice crystallization during warming. In some embodiments, sufficiently rapid and uniform rewarming can be achieved when the pulse width is between about 1 t_{d} and about 10 t_{d}. Pulse widths outside of this range may also be used and are within the scope of this description.

In some embodiments, the pulse width can be adapted to generate a minimum heating rate that is sufficient to rewarm the biomaterial with minimal biomaterial damage. The estimated overall warming rate or minimal warming rate can be estimated based on the total temperature increase in the selected biomaterial divided by the pulse width. Thus, the appropriate pulse width can be chosen to provide the selected rewarming rate.

In some embodiments, the pulse width can be adapted to generate a temperature change that is sufficient to rewarm the biomaterial with minimal biomaterial damage. The overall temperature change can be based on the total temperature increase desired in the biomaterial. Typically, the cryopreserved biomaterial will be rewarmed from a cryogenic temperature to a temperature above the melt temperature of the cryopreserved biomaterial, but below a temperature at which significant CPA toxicity or thermal damage is expected.

A variety of pulse widths can be applied to the conductor to achieve the selected heating rates for the rewarming of the cryopreserved biological composition. In some embodiments, the pulse widths can range between about 0.1 μs to about 10 seconds, or between about 1 μs to about 1 second, or between about 1 μs to about 100 milliseconds (ms). Pulse widths greater than about 10 seconds, or greater than about 60 seconds, or greater than about 2 minutes, or greater than about 5 minutes may also be used. Pulse widths outside this range are also within the scope of this description.

In some embodiments, the magnitude of the voltage pulse can vary during the rewarming process. The magnitude of the voltage pulse, the pulse width and/or the pulse waveform may vary during the rewarming process. Without being bound by any theory, it is thought that the electrical properties of a conductor can change with temperature, e.g., as the material warms from a cryogenic temperature. The voltage pulse may be adjusted to compensate for the changes in the electrical properties of the conductor as the temperature changes. In some embodiments, the voltage pulse may be controlled based on temperature feedback (or based on electrical feedback that changes with temperature) from the conductor or biomaterial. Temperature probe(s) may be placed within and/or adjacent to the biomaterial or conductor to provide a feedback loop between the temperature probe and the voltage pulse. The resistance of the conductor may also change in a repeatable manner with temperature and may be used as temperature feedback to control the response of the voltage pulse. In one embodiment, the feedback loop may be configured such that the temperature within the biomaterial does not exceed a selected temperature during rewarming by Joule heating. The voltage pulse may be adjusted as the selected temperature is approached in order to stay below the selected temperature. In one embodiment, the feedback loop may be configured to maintain a selected heating rate such that the voltage pulse may be adjusted to maintain the heating rate within a specified minimum and maximum range.

A variety of currents may be generated when a pulse is discharged from the generator. In some embodiments, the current generated is at least about 1 Amperes (A), or at least about 5 A, or at least about 10 A, or at least about 20 A, or at least about 40 A, or at least about 60 A, or at least about 80 A, or at least about 100 A, or at least about 500 A, or at least about 1000 A, or at least about 2000 A. Currents generated outside of this range are also within the scope of this description.

The methods described herein can convert a substantial amount of the input electrical energy into thermal energy. In some embodiments, at least about 70% of the input electrical energy is converted into thermal energy, or at least about 80% of the input electrical energy is converted into thermal energy, or at least about 90% of the input electrical energy is converted into thermal energy, or at least about 99% of the input electrical energy is converted into thermal energy.

In various embodiments, the characteristics of the discharged voltage pulse as described herein can be tuned to generate heat sufficient to rewarm across the heat diffusion length of the biomaterial with minimal biomaterial damage. In one embodiment, the thickness of the biomaterial can be the heat diffusion length generated by the Joule heating method. In one embodiment, half the thickness of the biomaterial can be the heat diffusion length generated by the Joule heating method. In some embodiments, the selected voltage pulse characteristics can be selected based on the thickness of the biomaterial. The description herein will be described in the context of the heat diffusion length correlating with the thickness of the biomaterial. It will be understood that dimensions other than the thickness of the biomaterial may be correlated with the heat diffusion length needed for uniform heating. The methods described herein can uniformly rewarm the cryopreserved biomaterial across the thickness of the biomaterial. Uniformly can be, for example, a temperature difference of no more than 175° C. across the biomaterial, or no more than 150° C. across the biomaterial, or no more than 100° C. across the biomaterial, or no more than 50° C. across the biomaterial, or no more than 25° C. across the biomaterial, or no more than 10° C. across the biomaterial.

The Joule heating methods disclosed herein can control rapid rewarming rates at the boundary of the resistive material, e.g. conductor. The rewarming rates of the biomaterial can vary and can be dependent on the heat diffusion length, e.g. thickness, of the biomaterial. In some embodiments, the cryopreserved biological composition can include biomaterial perfused with or suspended in a volume of the cryoprotective solution. The biomaterial or cryopreserved biological composition can have a heat diffusion length, e.g. thickness, of at least about 0.1 μm. Heat diffusion length is described in the context of thickness but it will be understood that the heat diffusion length can be other dimensions besides the thickness of the biomaterial. In some embodiments, the heat diffusion length of the biomaterial can be, for example, at least about 1 μm, or at least about 10 μm or at least about 0.1 mm, at least about 0.5 mm, at least about 1 mm, at least about 2 mm, at least about 5 mm. In the embodiments in which the biomaterial is perfused with the cryoprotective solution, the dimension listed above may be, in effect, the dimension of the biomaterial. In embodiments in which the biomaterial is suspended in a cryoprotective solution, the dimension may reflect a vessel retaining the cryopreserved biomaterial and/or a vessel in which the cryopreserved biomaterial was cooled or the dimension may be the size, e.g. the thickness, of a droplet of the cellular suspension.

In some embodiments, the method includes rewarming adherent cells and the adherent cells can include biomaterial with a thickness in the micrometer range, e.g. between about 1 μm and about 10 μm, or between about 2 μm and about 8 μm. In one embodiment, the method includes rewarming Drosophila embryos and the Drosophila embryos can include biomaterial with a thickness in the micrometer range, e.g. between about 10 μm and about 100 μm, or between about 30 μm and about 80 μm. In one embodiment, the method includes rewarming kidney slices and the kidney slices can include biomaterial with a thickness in the millimeter range, e.g. between about 0.5 mm and about 10 mm, or between about 1 mm and about 3 mm. Biomaterials with thickness outside of these ranges are also within the scope of this description.

In some embodiments, the warming rates of the biomaterial in the cryopreserved composition can be at least about 1×10³° C./min, at least about 1×10⁴° C./min, or at least about 1×10⁵° C./min, or at least about 1×10⁶° C./min, or at least about 1×10⁷° C./min, or at least about 1×10⁸° C./min. In some embodiments, the warming rates of the biomaterial in the cryopreserved composition can be no more than about 1×10¹⁴° C./min, or no more than about 1×10¹²° C./min, or no more than about 1×10¹⁰° C./min, or no more than about 1×10⁸° C./min, or no more than about 1×10⁷° C./min, or no more than about 1×10⁶° C./min, or no more than about 1×10⁵° C./min. In one embodiment, the warming rate of adherent cells can be about 6×10⁸° C./min. In one embodiment, the warming rate for Drosophila embryos can be about 4.2×10⁶° C./min. In one embodiment, the warming rates for kidney slices can be about 9×10⁴° C./min.

The method of rewarming the biomaterials described herein can be easily scalable to include biomaterials of larger sizes or large numbers of samples of the biomaterials. The conductor can be adapted to rewarm larger sizes of the biomaterial and/or a large number of samples of the biomaterials. In some embodiments, biomaterials of larger lengths and widths can be accommodated by increasing the size of the sheet or the mesh. The larger biomaterial can be heated at warming rates similar to smaller sizes if the heat diffusion length is similar. In some embodiments, the size of the biomaterial to be rewarmed or the number of samples rewarmed can be dependent on the size and configuration of the conductor if the heat diffusion length is similar. In some embodiments, the geometry of the conductor, e.g., a conductor with a 3D structure such as a porous our wound structure, can rewarm biomaterials that are within the structure. In these embodiments, heat diffusion length of between about 0.1 um and about 1 mm scale can be rewarmed and can maintain heating rates similar to a two dimensional structure.

In some embodiments, during scaling up the Joule heating platform, the size of the conductor may be increased. The impact of increasing the size of the conductor can vary. In some embodiments, the resistance may decrease or increase and can be dependent on the parameter of the conductor that is varied, e.g., length or width or cross-sectional area relative to the flow of electrical current. The magnitude of the voltage pulse can be adjusted to compensate for the increase or decrease in resistance. The pulse width may stay the same if the heat diffusion length of the biomaterial is similar to the heat diffusion length of the biomaterial prior to scaling up. FIG. 6A includes an approximate conversion on how the pulse width may depend on the thickness of the specimen. The voltage selected may depend on the conductor material and geometry chosen.

In some embodiments, the cryopreserved composition can include biomaterial perfused with or suspended in a volume having a linear dimension, e.g., length and/or width, of at least about 0.1 μm. In some embodiments, the thickness of the cryopreserved composition can be, for example, at least about 1 μm, or at least about 10 μm or at least about 0.1 mm, at least about 0.5 mm, at least about 1 mm, at least about 2 mm, at least about 5 mm, at least about 1 cm, at least about 2 cm, at least about 5 cm, at least about 10 cm, at least about 25 cm.

In various embodiments, the achievable warming rate may be limited by the heat diffusion length of the biosystem. In some embodiments, to achieve rapid and sufficiently uniform rewarming, the estimated overall warming rates for biosystems with about 10 μm, about 100 μm, about 1 mm, and about 1 cm heat-diffusion lengths can be about 2×10⁸, about 2×10⁶, about 2×10⁴, and about 2×10²° C./min, respectively.

In some embodiments, the methods described herein can allow the scaling up for high throughput cryopreservation. With almost 100% of input energy converted into thermal energy for rewarming, Joule heating is a scalable platform technique. In one embodiment, a 290 V, 1 ms voltage pulse can be used to rewarm a 15 cm×1 cm SS mesh (i.e., 1.4Ω) loaded with Drosophila embryos. To scale up the area of SS mesh by 9-fold (i.e., 45 cm×3 cm), the resistance remains the same and the voltage only needs to increase by 3-fold (i.e., 870 V) to achieve the same warming rates (equations 1 and 6 below in Examples). Joule heating can allow high throughput cryopreservation while maintaining rapid warming rates.

The method of the present description can include cooling the biological specimen. In some embodiments, this disclosure can include a method of cryopreserving a biomaterial perfused in and/or suspended in a cryoprotective solution and cooling the biomaterial to a cryopreserved state. In some embodiments, the biomaterial can be placed on the conductor in direct thermal contact prior to cooling to a cryogenic temperature. In some embodiments, the biomaterial can be placed on the conductor in thermal contact after the biomaterial has been cooled to a cryogenic temperature. In some embodiment, there can be an intermediate layer between the conductor and the biomaterial as described herein.

In various embodiments, the biomaterial may be cooled to a temperature of no more than 0° C. such as, for example, no more than −20° C., no more than −40° C., no more than −80° C., no more than −100° C., no more than −120° C., no more than −130° C., no more than −140° C., no more than −150° C., no more than −160° C., no more than −170° C., no more than −180° C., no more than −190° C., or no more than −196° C. In some embodiments, suitable temperatures can include a minimum temperature of no less than −196° C., or no less than −150° C. In one embodiment, a suitable temperature for the cryopreserved biomaterial may be the boiling point of nitrogen, about −196° C.

The cryoprotective agent(s) can include any material suitable for the cryopreservation of biomaterials. Exemplary suitable cryoprotective agents include, for example, combinations of alcohols, sugars, polymers, organic compounds and ice blocking molecules that alter the phase diagram of water and allow a glass to be formed more easily (and/or at higher temperatures) while also reducing the likelihood of ice nucleation and growth during cooling or thawing. The cryopreservative agents may be used alone or in a combination such as in cocktails. In the case of vitrification solutions, exemplary cryopreservative cocktails are reviewed in Fahy et al. Cryobiology 48(1):22-35, 2004, incorporated herein by reference. The cryoprotective agents can include, for example, one or more of the following: dimethyl sulfoxide, glycerol, propylene glycol, ethylene glycol, formamide, sucrose, glucose, glucose analogs, trehalose, raffinose, polyvinylpyrrolidone, and/or other polymers (e.g., ice blockers and/or anti-freeze proteins).

In some embodiments, the methods described herein can result in rapid warming rates generated by Joule heating, facilitating use of lower CPA concentration and thus reducing toxicity. This can improve the cryopreservation of numerous biosystems that are intolerable to the toxicity of high CPA concentrations such as, for example, aquatic species, mosquito larvae/embryos, cell therapy products and mammalian embryos and oocytes.

In some embodiments, one or more cryoprotective agent(s) may be present in the cryoprotective solution at a molarity of no more than 9M, such as, for example, no more than 6 M, no more than 5 M, no more than 4 M, no more than 3 M, no more than 2 M, no more than 1 M, no more than 900 mM, no more than 800 mM, no more than 700 mM, no more than 600 mM, no more than 500 mM, or no more than 250 mM.

The biomaterial in the cryoprotective composition described herein may be cooled to a cryogenic temperature at a rate effective for cryopreservation. Cooling rates can promote vitrification of the biomaterial. In some embodiments, the biomaterial may be cooled at a minimum rate of at least 0.1° C. per minute (° C./min) such as, for example, at least 0.5° C./min, at least 1° C./min, at least 2° C./min, at least 5° C./min, at least 10° C./min, at least 15° C./min, at least 20° C./min, at least 25° C./min, at least 30° C./min, at least 40° C./min, at least 50° C./min, at least 60° C./min, at least 70° C./min, at least 100° C./min, at least 1000° C./min, multiple thousands ° C./min, or multiple millions of ° C./min. In some embodiments, the cooling rate may be within a range of cooling rates having endpoints defined by any minimum cooling rate listed above and any maximum cooling rate listed above that is greater than the minimum cooling rate. In embodiments involving larger systems, the cooling process can involve use of a high pressure freezing vial as described by Fahy et al. Cryobiology 48(2):157-178, 2004. Added pressure—e.g., up to 1000 atm—can reduce the ability of ice to nucleate and grow within the sample during cooling.

In some embodiments, the biomaterial perfused with or suspended in the cryoprotective composition may be plunge cooled in the cryogenic coolant, e.g. liquid nitrogen, prior to placing on the conductor that is at a cryogenic temperature.

In some embodiments, the biomaterials perfused with or suspended in the cryoprotective composition may be placed in thermal contact with the conductor. The conductor with the biomaterial in the cryoprotective composition may be plunge cooled in a cryogenic coolant to a cryogenic temperature.

The various embodiments will be further described by reference to the following examples, which are offered to further illustrate various embodiments of the present description. It should be understood, however, that many variations and modifications may be made while remaining within the scope of the various embodiments.

### EXAMPLES

### Methods

### Voltage, Current and Resistance Measurement

A commercial pulse generator (ECM® 830, BTX® Harvard Apparatus, Holliston, MA) was used in this study. The low voltage (LV, voltage ≤500V) mode was used, with adjustable voltage and pulse width. Stainless steel (SS) 304 was chosen for the conductor material. This included a SS sheet, with thickness of 12 μm, and a SS mesh, with wire diameter of 30 μm and aperture (i.e., pore size) of 38 μm. The current and voltage were measured by a current probe (P6021A, Tektronix) and voltage probe (Enhancer 3000, Harvard Apparatus), respectively. An oscilloscope (DS1M12, USB Instrument) was used to record the measured current and voltage. The sample rate of this oscilloscope was up to 1 MS/s (i.e., 10⁶ samples per second). The band width of this oscilloscope was 250 kHz. For non-sinusoidal waveforms (i.e., square waves, pulses, etc.), a bandwidth of 10 times the fundamental frequency (1/pulse width in our case) would be appropriate to capture the rise of signal. Therefore, for pulse widths larger than 40 μs, this oscilloscope provided reliable measurements. To accurately measure the resistance of a SS sheet and mesh with certain dimensions, the voltage was altered to establish the voltage-current correlation where the resistance can be calculated. The effective electrical conductivity (σ) of the SS mesh or sheet was calculated based on their dimensions and measured resistance.

### Temperature Measurement of Commercial Thin Film Resistor

The commercial thin film resistors (150Ω) with electrically insulated surface coating were used to measure the temperature during Joule heating. An ultrafine gauge type T thermocouple (COCO-002, OMEGA) was attached to the surface of a resistor. The temperature was recorded using an oscilloscope (DS1M12, USB Instrument). The resistor was submerged in liquid nitrogen during the entire measurement process. One electric pulse (5 ms) was applied, and temperature was recorded at a resolution of 1 ms. The warming rate was calculated from the temperature zone −170° C. to −10° C. in this study unless otherwise noted. Different voltages ranging from 100 V to 300 V were tested. Note that when a thermocouple was directly in contact with the SS sheet or mesh, the applied voltage pulse interfered with the thermocouple signal. Therefore, the temperature of the SS sheet or mesh was not directly measured.

### Energy Delivery and Specific Absorption Rate (SAR)

A single pulse was used in this study. For the low voltage mode of the pulse generator, the capacitance is 4 mF, maximum current is 500 A, maximum voltage is 500 V, and the pulse width range is 10 μs~10 s. The thermal energy generated by the Joule heating per pulse (assuming a square wave) can be calculated as below:

$$Q = {\int }_{0}^{pw} \frac{{U}^{2}}{R} dt = {\int }_{0}^{pw} \frac{{( {U}_{0} {e}^{- t / RC} )}^{2}}{R} dt = \frac{{{CU}_{0}}^{2}}{2} ( 1 - {e}^{- \frac{2 pw}{RC}} ) \qquad ( 2 )$$

Or equivalently:

$$Q = {\int }_{0}^{pw} \frac{{U}^{2}}{R} dt = {\int }_{0}^{pw} \frac{{( {U}_{0} {e}^{- t / RC} )}^{2}}{R} dt = \frac{{{CU}_{0}}^{2}}{2} ( 1 - {e}^{- \frac{2 pw}{RC}} )$$

Where Q is the energy generated by Joule heating, R is the resistance of the electrical conductor in contact with the biosystems, C is the capacitance of the capacitor, U is voltage, I is current, U₀ and I₀ are the peak voltage and current, t is time, pw is pulse width. For simplicity, R is considered as a constant in equations 1-2.

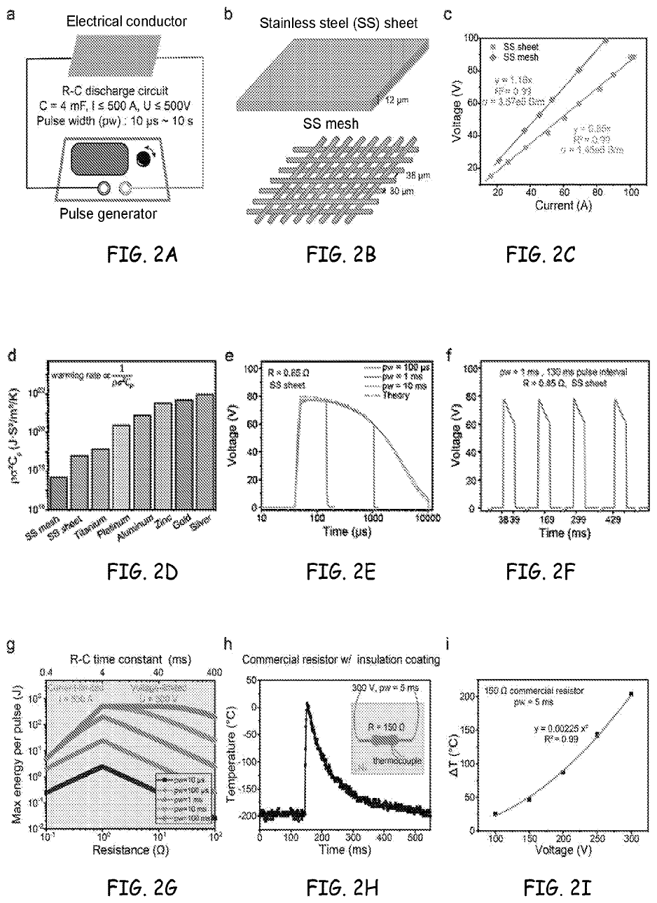

To calculate the maximum energy delivered per pulse shown in FIG. 2G, equation 1 was used for R≥1Ω and U₀ set to 500 V; equation 2 was used for R≤1Ω and I₀ set to 500 A.

To estimate the warming rate of the electrical conductor, the temperature change within the pulse width can be calculated as below:

$$Q = \frac{{CR}^{2} {{I}_{0}}^{2}}{2} ( 1 - {e}^{- \frac{2 pw}{RC}} ) = {mC}_{p} Δ T \qquad ( 4 )$$

$$R = \frac{L}{σ A} \qquad ( 5 )$$

$$m = ρ LA \qquad ( 6 )$$

$$Δ T = \frac{{{CI}_{0}}^{2}}{2} ( 1 - {e}^{- \frac{2 pw}{RC}} ) \frac{{R}^{2}}{{mC}_{p}} ∝ \frac{{R}^{2}}{{mC}_{p}} = \frac{L}{{A}^{3}} \frac{1}{{σ}^{2} ρ {C}_{p}} \qquad ( 7 )$$

Where, for the electrical conductor, m is mass, ρ is density, L is length, A is cross section area, σ is electrical conductivity, Cₚ is heat capacity, ΔT is the temperature change, as shown in FIG. 2D.

The specific absorption rate (SAR, W/m³), the heat source term used in the heat transfer modeling, is estimated as shown below:

$$SAR = \frac{{( {U}_{0} {e}^{- t / RC} )}^{2}}{RV} \qquad ( 8 )$$

$$R = {R}_{0} ( 1 + α ( T - {T}_{0} ) ) \qquad ( 9 )$$

Where, V is the volume of the electrical conductor, R₀ is the resistance at 293 K, T₀ is 293 K, α is the temperature coefficient of resistivity (e.g. 0.94×10⁻³ K⁻¹ for stainless steel).

### Adherent Cells Cryopreservation

Human dermal fibroblasts (HDFs) were cultured in Dulbecco's modified Eagle media (DMEM) that contained 10% fetal bovine serum (Thermo Fisher Scientific, Waltham, MA) and 1% penicillin streptomycin (Sigma, St. Louis, MO) at 37° C. under 5% CO₂. The HDFs were purchased from ATCC (PCS-201-012) and stored in a liquid nitrogen Dewar flask that is maintained in the lab. Prior to adding HDFs, the SS sheet was sterilized by ethanol spray and UV irradiation. After adding cells to the SS sheet, overnight incubation was allowed for cell attachment before cryopreservation.

For cryopreservation, various dilutions (25%, 50%, 75% and 100%) of VS55 were used as the CPA. VS55 is a cryopreservation solution composed of 3.1 M dimethyl sulfoxide (DMSO), 2.2 M propylene glycol, and 3.1 M formamide (for a total of 8.4 M) in a carrier solution. CPA loading and unloading were performed in a stepwise manner with 3 min for each step at 4° C. as previously reported (Gao, Advance Science, 2020). The relative CPA concentration for each loading/unloading step was 18.75%, 25%, 50%, 75% and 100% of the final VS55 concentration. At the end of CPA loading, the excess CPA solution on the SS sheet was removed. The SS sheet with attached cells was plunged into liquid nitrogen for cooling. For convective warming, the SS sheet was plunged into the unloading solution at 4° C., followed by stepwise CPA unloading. For Joule heating, the SS sheet was connected to the pulse generator, then brought out of liquid nitrogen and immediately rewarmed by the voltage pulse, followed by stepwise CPA unloading. Using a stopwatch, we measured that it took 0.25~0.4 s to bring out the SS sheet from liquid nitrogen until the application of the Joule heating pulse. Assuming a convective heat transfer coefficient of 10 W/m²/K, heat transfer modeling suggested that the temperature of cells increased to −173~−161° C., still below the glass transition temperature. The tested pulse widths included 10 μs, 100 μs and 1 ms.

To measure the convective cooling and warming rates using the SS sheet, an ultrafine gauge type T thermocouple (COCO-002, OMEGA, Ralston, NE) was attached to the surface of the SS sheet. The temperature was recorded using an oscilloscope (DS1M12, USB Instrument) when the SS sheet was plunged into liquid nitrogen for cooling, and into water bath for rewarming.

Hoechst and propidium iodide (PI) were used to assess cell viability by membrane integrity. Fluorescent microscope images were taken under 4× by an Olympus IX50 microscope. Fiji (ImageJ) was used to quantify the viability.

### Drosophila Embryos Cryopreservation

A wildtype stock (named “WC1b” as previously reported (Zhan, Nature Communications, 2021)) of Drosophila melanogaster was used in this study. The flies were maintained at room temperature. Following the previously reported protocol, embryos were collected over a 1 hour period on a grape juice plate smeared with yeast paste, and incubated in a 20° C. incubator until reaching 22 hrs old. The embryos were then dechorionated and permeabilized for CPA loading. The first CPA loading step involved incubation in 13% EG at room temperature for 25 min. Next, the embryos were transferred to various dehydration CPAs on ice for 9 min. In this study, four different dehydration CPAs were tested including 21% EG+9% sorbitol, 27% EG+9% sorbitol, 33% EG+9% sorbitol, and 39% EG+9% sorbitol. The dehydrated embryos were transferred to the SS mesh and excess CPA solution was wicked away. The SS mesh with the embryos were quickly plunged into liquid nitrogen. The embryos remain attached to the SS mesh in liquid nitrogen due to the presence of residual CPA solution between the embryo and SS mesh. To confirm if the embryos were vitrified, the SS mesh with embryos were placed in liquid nitrogen vapor and observed under a dissecting microscope. Images of embryos in liquid nitrogen were taken using a C-mount microscope camera (MU1000, AmScope).

For convective warming, the SS mesh was rapidly plunged into 30% sucrose solution. After a few second, the SS mesh and embryos were transferred to 15% sucrose for 3 min, followed by washing in isotonic buffer for 20 min for CPA unloading. For Joule heating, the SS mesh was connected to the pulse generator, brought out of liquid nitrogen and immediately rewarmed by a voltage pulse. The tested pulse width was 1 ms. The embryos were placed in 15% sucrose for 3 min and isotonic buffer for 20 min for CPA removal.

After CPA removal, the embryos were transferred to a 35 mm petri dish with 1 ml Schneider medium for further development. On the next day, the hatched larvae were counted and transferred to the food vials (15~95 mm shell vial) for development into adults. The hatch rate (i.e., embryo to larva rate) was calculated using the ratio of hatched larvae to total number of embryos. After 15 days at room temperature, the adult rate (i.e., larva to adult) was calculated using the ratio of emerged adults to total number of larvae placed in the vials.

### Joule Heating High-Speed Video of Drosophila Embryos

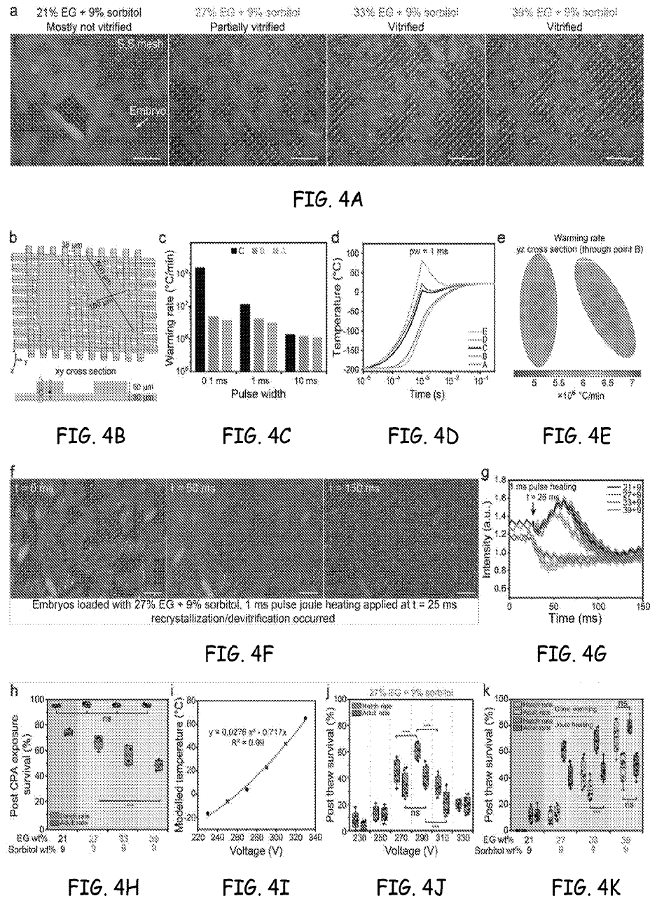

To observe ice formation during the rapid Joule heating process, a high speed camera (MEMRECAM Q1v, nac Image Technology) was used to record the rewarming of Drosophila embryos on the SS mesh at 3000 frames per second (fps). Drosophila embryos were the only model system visualized in this manner because 1) the frame rate of the high speed camera is not fast enough to capture the rewarming of HDF cells (a 10 μs pulse was used for rewarming); and 2) it is challenging to visualize the inside of kidney slices where the ice is expected to occur (1.2 mm thick). To capture more embryos, the high speed camera was mounted to a dissecting microscope. Four different dehydration CPAs including 21% EG+9% sorbitol, 27% EG+9% sorbitol, 33% EG+9% sorbitol, and 39% EG+9% sorbitol were tested. A customized MATLAB script was used to extract the time-dependent grayscale intensity of embryos during the Joule heating process. Analysis of the video was performed and provided as data in FIGS. 4A-4E.

### Kidney Slices Cryopreservation

Kidneys were procured from 3-month-old Sprague-Dawley rats, following procedural protocol approved by the Institutional Animal Care and Use Committee (IACUC) at the University of Minnesota. Heparin (1000 IU) was injected intraperitoneally to the rat, 1-2 min before the euthanasia. The kidneys were removed immediately after euthanasia. The kidneys were rinsed briefly with PBS and placed in UW solution for transportation. Kidney cortex slices with 1.2 mm thickness were prepared using a 6 mm punch biopsy and a custom modified Stadie-Riggs tissue slicer. The slices were incubated with 10% alamarBlue at 37° C. for 2 hrs to obtain the fluorescent readings before treatment. Three CPA concentrations (50% VS55, 62.5% VS55 and 75% VS55) were tested. CPA loading was performed on ice in a stepwise manner with 10 min step duration as previously reported (Han, Advanced Healthcare Materials, 2020). At the end of CPA loading, the kidney slice was sandwiched by a pre-folded SS mesh strip and the excess CPA solution was removed. A light plastic forceps was modified such that the gap at the tip of the forceps was 1.2 mm. The modified forceps was used to hold the SS mesh and kidney slice, maintaining close contact between them when plunged into liquid nitrogen. The forceps were removed after cooling. To rewarm the sample, the SS mesh was first connected to the pulse generator and brought out of liquid nitrogen. Before applying the voltage pulse, a modified plastic forceps at room temperature was used to hold the SS mesh and kidney slice to ensure close contact between the mesh and tissue during Joule heating. After rewarming, the tissue was subjected to stepwise CPA unloading on ice.

The convective warming was performed using a cryovial as reported in the literature (de Graaf, Cryobiology, 2007). Briefly, after CPA loading, the kidney slice was transferred to a cryovial with 1 ml final concentration CPA solution. The cryovial was plunged into liquid nitrogen for cooling and plunged into a room temperature water bath (~2 min) for rewarming.

Viability of the kidney slices was assessed by incubating the tissue with 10% alamarBlue at 37° C. for 2 hrs. This 2 hrs incubation provides enough time for alamarBlue to penetrate the 1.2 mm tissue as suggested by previous study (Han, Advanced Healthcare Materials, 2020). A plate reader (Synergy HT, BioTek, Winooski, VT) was used to read the fluorescence of the supernatant. Kidney slices were incubated with alamarBlue before and after cryopreservation to obtain the corresponding fluorescence readings. The viability was reported as the ratio of those two readings from the same slice to avoid sample to sample variations.

Histology was performed on kidney slices from various treatment conditions including positive control (no treatment), Joule heating (290 V, 100 ms) of 75% VS55 loaded kidney slices, air warming of 75% VS55 loaded kidney slices (i.e., negative control), and convective warming of 75% loaded kidney slices. Kidney slices were immediately transferred to 10% neutral buffered formalin after treatment and paraffin embedded within 48 hrs. Sections of ~5 μm thickness were used for hematoxylin & eosin (H & E) staining and imaging.

### Heat Transfer Modeling

The heat transfer was modelled using COMSOL 5.5 for the Joule heating of adherent cells, Drosophila embryos and kidney slices. The governing equations for the SS and biosystems are given as follow:

$${ρ}_{S} {C}_{p \_ S} \frac{\partial {T}_{S}}{\partial t} = {k}_{S} {∇}^{2} {T}_{S} + SAR \qquad ( 10 )$$

$${ρ}_{B} {C}_{p \_ B} \frac{\partial {T}_{B}}{\partial t} = {k}_{B} {∇}^{2} {T}_{B} \qquad ( 11 )$$

Where the subscript S represents SS sheet or mesh, subscript B represents biosystem, SAR is calculated from equations 7-8, ρ is density, Cₚ is heat capacity, k is thermal conductivity, T is temperature. For various biosystems (adherent cells, Drosophila embryos, kidney slices), in the absence of experimental data on the material properties, the thermal properties of relevant samples were used as previously reported in the literature. Specifically, temperature dependent density of VS55 was used for all biosystems. The reported temperature dependent thermal conductivity of 6 M glycerol-laden porcine liver was used as the estimation for all biosystems. For heat capacity, similar thermal properties have been reported between CPA solutions and CPA-loaded biological samples, so we used the experimentally measured values for VS55 as the approximation for all biosystems. It is expected that our simulation provides an accurate representation of the heat transfer processes involved based on these material property estimations. For the SS, the temperature dependent thermal conductivity and specific heat were obtained from National Institute of Standards and Technology.

The boundary condition for the outer surfaces was natural convection at room temperature (293 K). The convective heat transfer coefficient (h_{conv}) was assumed 10 W/m²/K. For the adherent cell modeling, the thickness of the cell monolayer was set to 4 μm as reported in the literature. The thickness of Drosophila embryos was 50 μm as previously reported.

For different biosystems, we selected the geometry to capture the key dimensions affecting the heat transfer and minimize the cost of computational resources. For adherent cells, a 2D area (50 μm width, capturing the thickness of the cell monolayer and S.S. sheet, FIG. 15) from the cross section perpendicular to the length of S.S. sheet was used. A 3D geometry (FIG. 15) was used as the sizes of a Drosophila embryo (500*180*50 μm) and S.S mesh (wire diameter 30 μm, aperture size 38 μm) are on the same order of magnitude. For kidney slides, a 2D area (1 mm width, FIG. 15) from the cross section perpendicular to the diameter of the kidney slices was used. The detailed dimensions and mesh (i.e., finite element method) can be found in FIG. 15.

### Kidney Slices CPA Loading and Thermal Injury Modeling

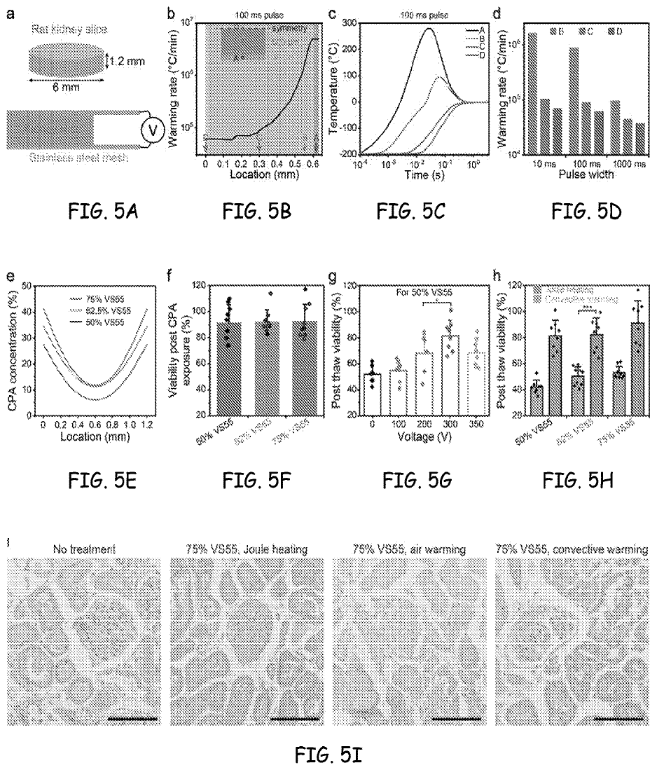

The Fick's second law shown below was used to model the CPA diffusion into the kidney slices:

$$\frac{1}{D} \frac{\partial C}{\partial t} = \frac{{\partial }^{2} C}{\partial {x}^{2}} \qquad ( 12 )$$

Where Cis CPA concentration, t is time and D is the effective diffusion coefficient, x is the location (depth) inside the tissue. D=6.5×10⁻¹¹ m²/s as reported in previous study was used (Han, Advanced Healthcare Materials, 2020). COMSOL 5.5 was used for the numerical simulation. Protocols with different final CPA concentrations including 50% VS55, 62.5% VS55 and 75% VS55 were modelled. The distributions of CPA concentration after loading were plotted in FIG. 5E.

To assess the potential thermal injury to kidney slices during Joule heating (i.e., point B in FIG. 5C), the resultant thermally induced injury can be estimated assuming a first order kinetic process using Arrhenius model:

$$S = 1 - {e}^{- {\int }_{0}^{t} Ae - ( \frac{Δ E}{RT} ) dt} \qquad ( 13 )$$

Where S is the thermally induced injury, ΔE is activation energy, A is frequency factor, R is universal gas constant (8.314 J/mole/K), T is temperature (in Kelvin). Activation energy was 168.42 kJ/mole, frequency factor was 2.53×10²⁴ s⁻¹, obtained from the literature.

### Statistics

Experimental data was presented as mean±s.d. unless otherwise specified. Sample size (n) for experimental data was included in the figure descriptions. For plots with two dependent variables, for instance, hatch rate and adult rate for Drosophila embryo experiments, multivariate analysis of variance (MANOVA) and Tukey's post hoc were used for statistical analysis. A paired one-way ANOVA and Tukey's post hoc was used for statistical analysis of other experimental results. The p values >0.05 were considered statistically non-significant (ns). The p values <0.05 were considered statistically significant, with * p<0.05, **p<0.01, ***p<0.001.

### Results

### Design of Biosystem Rewarming Using Joule Heating

Rapid rewarming of biosystems in a simple manner using a boundary heat source (i.e., adjacent to the biosystems) by Joule heating was achieved. The biosystems were in thermal contact with an electrical conductor and rewarmed by heat conduction (FIG. 1A). The rates achievable within the system are limited by the rate of heat diffusion within the system, with slower warming rates experienced by the biosystems at points or locations further from the heat source. To represent different thicknesses, model biosystems including adherent cells (4 μm), Drosophila embryos (50 μm) and rat kidney slices (1.2 mm) (FIG. 1B) were included. During cryopreservation, excess CPA solution was removed after CPA loading to reduce the thermal mass and improve the cooling/warming rates (Zhan, Nature Communications, 2021). Rapid cooling was achieved by direct plunging into liquid nitrogen (FIG. 1A). The electrical conductor was then connected to a voltage pulse generator for rewarming. Adjustable warming rates across a wide range can be achieved by using voltage pulse generators with a wide range of pulse widths (microseconds to seconds and longer) and voltages (several volts to thousands of volts and higher).

### Characterization of Joule Heating in the RC Discharge Circuit

To generate controllable rapid Joule heating, a resistor-capacitor (RC) discharge circuit using a voltage pulse generator (ECM® 830, BTX® Harvard Apparatus) that can produce pulse widths ranging from 10 μs to 10 s was selected. The capacitor was charged to a preset voltage and energy was dissipated to the resistor (i.e., electrical conductor in FIG. 2A) via Joule heating within the preset pulse width during the discharge phase. The voltage and pulse width were adjusted to characterize the range of performance. The low voltage (LV) mode of the pulse generator was used, with maximum voltage of 500 V and maximum current of 500 A. The capacitance was 4,000 μF. To select the electrical conductors, the warming rate was estimated by assuming the ohmic energy dissipation is converted to thermal energy of the conductor within the voltage pulse (equations 3-6). Under the same electrical current, material dimensions and pulse width, it was found that warming rate is inversely correlated to three material properties including density (ρ), electrical conductivity (σ) and heat capacity (Cₚ). As shown in FIG. 2D, among the commonly used metal materials, stainless steel (SS) provides the highest warming rate (i.e., lowest value of ρ*σ²*Cₚ). In addition, the low cost and good biocompatibility of SS have been well documented. A SS sheet (12 μm thickness) and mesh (wire diameter 30 μm, aperture size 38 μm) were used in this study as shown in FIG. 2B. The SS sheet permits cell attachment and was used for cryopreservation of adherent cells (i.e., human dermal fibroblast in this study). The SS mesh allows easy removal of excess CPA solution to minimize the thermal mass for improved cooling and warming of the biosystems (i.e., Drosophila embryos and kidney slices in this study). Considering the woven format of the SS mesh, the “effective” electrical conductivity was obtained from voltage-current (U-I) measurements to facilitate numerical modeling of the Joule heating process (equations 1-10). Specifically, a section SS mesh (12 cm length by 1 cm width) was subjected to different voltages and the current was measured (FIG. 2C). The resistance (R) and electrical conductivity (σ) were obtained from the U-I correlation. The same measurement was performed on a SS sheet (15 cm length by 1 cm width). The measured electrical conductivity of SS sheet was 1.45×10⁶ S/m, matching with the literature values. The SS mesh showed a lower effective electrical conductivity of 3.57×10⁵ S/m.

The voltage profile and energy delivery ability of the pulse generator was investigated. The SS sheet (R=0.85Ω) was subjected to 80 V with different pulse widths (100 μs, 1 ms and 10 ms). The RC time constant of the discharging capacitor was 3.4 ms (i.e., 0.85 Ω×4 mF). The recorded voltage profile showed an exponential decay within the pulse width and good agreement with theoretical estimates (FIG. 2E). At the timepoint of 5*(RC time constant), the voltage drops to 0.7% (i.e., e⁻⁵) of the peak value, suggesting that the feasible range of the pulse width is resistance dependent. Although one single voltage pulse was used in this study for cryopreservation, it was shown that multiple pulses with a preset interval can be realized in high fidelity using this pulse generator (FIG. 2F). The decaying voltage implied potentially decreasing warming rate during the pulse when pulse width is larger than the RC time constant, which can be readily addressed by applying a square-wave voltage pulse in the future. To understand the maximum energy each voltage pulse can deliver for different pulse widths, the resistance of 1Ω as the boundary between current-limited and voltage-limited regions was identified. For example, given the pulse generator has a maximum voltage of 500 V and maximum current of 500 A, for a conductor with <1Ω resistance, the current limit of 500 A will be reached before the voltage limit of 500 V. According to equations 1-2, the maximum energy per pulse was plotted with the resistance and pulse width as shown in FIG. 2G. For a given resistance, the max energy per pulse increases with pulse width and reaches the plateau once the pulse width exceeds 5*(RC time constant) (upper x axis in FIG. 2G). This is due to the completion of capacitive discharge before the pulse width ends. In addition, it was found that, compared to other resistance values, the resistance of 1Ω produced the highest energy for different pulse widths when complete discharge of the capacitor was not achieved (i.e., pulse width <5*(RC time constant)). When complete discharge occurred (i.e., the horizontal portion of the green line in FIG. 2G), the max energy per pulse remained the same and the resistance can vary from 1Ω to ~10Ω. These findings can guide the selection of resistance values for cryopreservation of various biosystems using different pulse widths, when operating within the limits of the generator used in this study. For example, to achieve optimal energy delivery, for pulse widths <10 ms, a resistance value around 1Ω is preferred (orange line in FIG. 2G); for the pulse width of 100 ms, the optimal resistance value is more flexible (green line in FIG. 2G). Furthermore, the maximum energy delivered from the pulse generator per pulse was 500 J as shown in FIG. 2G, which matched with the energy stored in a fully charged capacitor of

$$500 V ( i . e . , \frac{1}{2} {C V}^{2} )$$

To validate the Joule heating performance, the temperature change of the conductor (thin film resistor) after one voltage pulse was measured. When the thermocouple was in contact with the SS sheet or mesh for direct temperature measurement, the applied voltage pulse interfered with the thermocouple output voltage signals which are ~50 μV/° C. Therefore, only the temperature of the commercial thin film resistor with an electrically insulated coating was measured. Specifically, an ultrafine gauge thermocouple was attached to the surface of a 150Ω thin film resistor which was connected to the pulse generator. The resistor and thermocouple were submerged in liquid nitrogen during the entire measurement. FIG. 2H presented the measured temperature profile with 1 ms resolution when a 300 V, 5 ms voltage pulse was applied. It was observed that the temperature of the resistor rapidly increased from −196° C. to 7.6° C. within 5 ms (warming rate of 2.4×10⁶° C./min), and then decreased to −196° C. These results demonstrated the physical feasibility of achieving rapid warming in a cryogenic object with Joule heating using a pulse generator. Further, it was shown that the temperature increase can be controlled with high consistency by adjusting the pulse voltage (FIG. 2I), which was found to be proportional to the square of the voltage, as suggested by the theoretical calculation (equation 1). Finally, heating can also be accomplished using a sandwich system (i.e., the conductor can be constructed in a multidimensional configuration) thereby increasing thickness and homogeneity in thicker systems.

### Joule Heating for Adherent Cell Cryopreservation

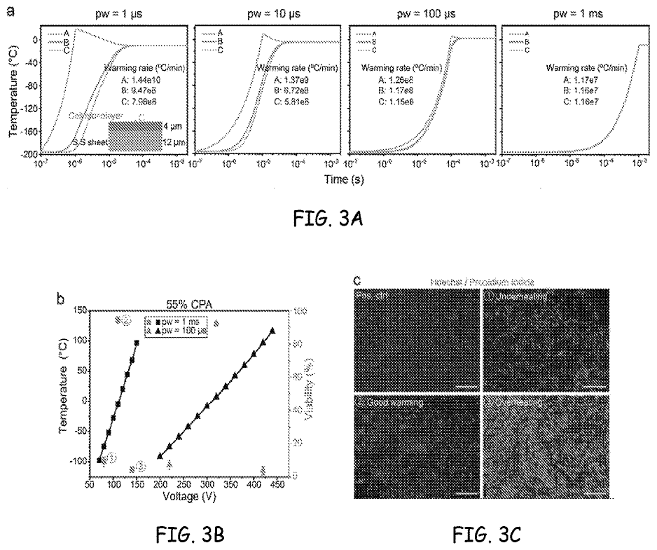

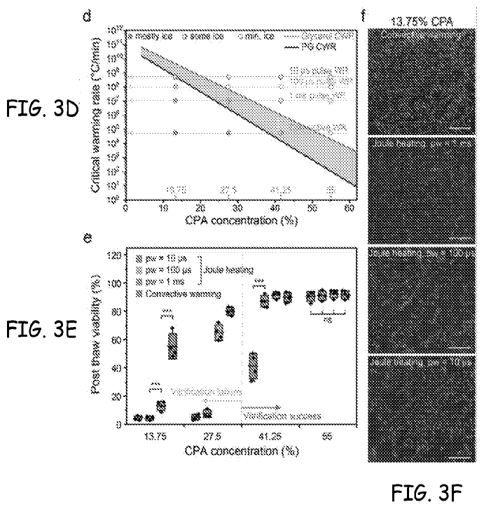

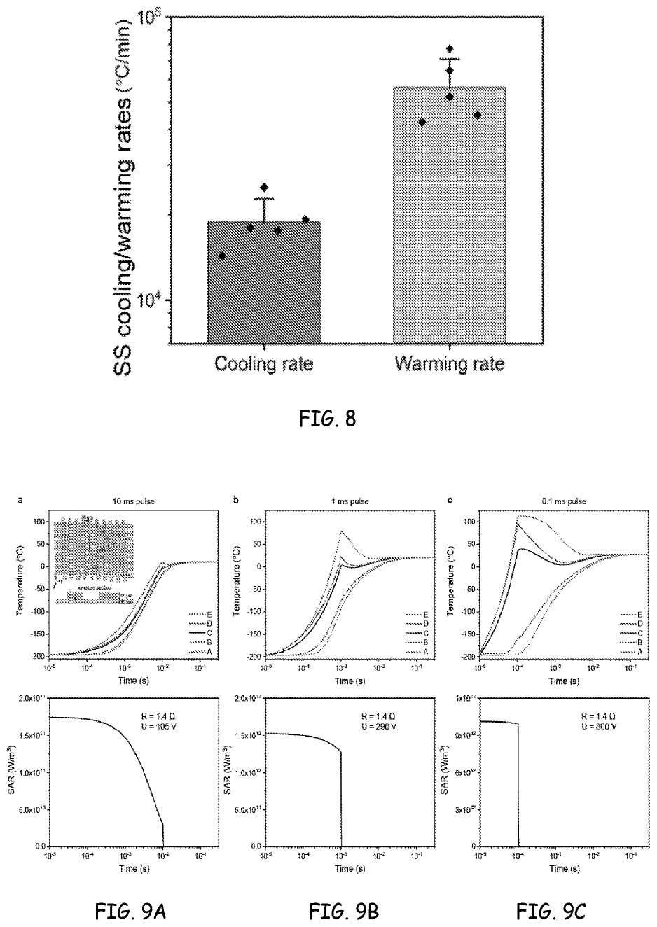

After physical characterization of the Joule heating approach, application for adherent cell cryopreservation was explored. Human dermal fibroblasts (HDFs) were seeded on a SS sheet (12 μm thickness, 15 cm length, 1 cm width, R=0.85Ω) and incubated overnight for attachment. While cryopreservation of HDFs is routinely achieved through traditional slow-cooling methods, they were chosen as a well-characterized model system to study the cryopreservation response at the single cell scale. Adherent cells are ultrathin (~4 μm), therefore could achieve ultrarapid heat diffusion and warming rates. Heat transfer simulations to estimate the warming rates for different pulse widths (1 μs, 10 μs, 100 μs and 1 ms) were performed. The RC time constant is 3.4 ms, exceeding the tested pulse widths. The specific absorption rate (SAR) was 7×10¹⁴, 5.9×10¹³, 6.6×10¹² and 7.8×10¹¹ W/m³ for 1 μs, 10 μs, 100 μs and 1 ms Joule heating in the modeling, respectively (Equation 7-8, FIG. 7). In FIG. 3A, the simulated temperature profile and warming rate at the middle of the SS sheet (point A), middle of adherent cells (point B) and top of adherent cells (point C) are presented. As pulse width increased from 1 μs, 10 μs, 100 μs to 1 ms, the predicted warming rate of point A decreased from 1.4×10¹⁰, 1.4×10⁹, 1.3×10⁸ to 1.2×10⁷° C./min, and the predicted warming rate at point B decreased from 9.5×10⁸, 6.7×10⁸, 1.2×10⁸ to 1.2×10⁷° C./min. In addition, the predicted temperature differences within the cells, and between the SS sheet and cells decreased with increasing pulse width. The warming rate of the conventional convective warming method was measured to be 5.6×10⁴° C./min (FIG. 8). The heat diffusion time (t_{d}) across the cell is estimated to be 8 μs. For a 1 μs pulse (i.e., <t_{d}), although the SS sheet was rewarmed 10× faster than a 10 μs pulse, the middle and top of cells (point B and C) were only rewarmed 1.4× faster, due to the thermal conductive effects through the system as expected (FIG. 3A). In contrast, as pulse width increased beyond I_{d}, the warming rate of the SS sheet more closely matched that of the cells.

To investigate the effect of warming rates on cell viability, cryopreservation of adherent HDFs using 10 μs, 100 μs and 1 ms pulse Joule heating, and conventional convective warming approaches was tested. Different dilutions (25%, 50%, 75% and 100%) of VS55 were used. For example, 100% VS55 is 55 wt % CPA (i.e., 8.4 M) and 25% VS55 is 13.75 wt % CPA (i.e., 2.1 M). The CPA was loaded/unloaded in a stepwise manner on ice. After CPA loading, excess CPA solution was removed by wicking methods prior to cooling. The cooling rate (i.e., repeatable plunge cooling procedure) was kept the same (1.9×10⁴° C./min, FIG. 8) for all conditions. To optimize the voltage for different pulse widths, the final temperatures of the cells after Joule heating were modelled (FIG. 2B). A higher voltage was required a the shorter pulse width to achieve the same final temperature (FIG. 2B). For 100 μs and 1 ms pulse, VS55 was used to test the cell viability in the following conditions: 1) underheating, final temperature <−60° C.; 2) target warming temperature ~0° C.; 3) overheating, final temperature >60° C. Hoechst/Propidium iodide (PI) membrane integrity dye was used as the viability indicator. As shown in FIGS. 3B-C, high viability was obtained for the target warming condition, while the viability was very low for both underheating and overheating conditions. Using the optimized voltages, the CPA concentration was also varied to investigate the impact on viability. FIG. 3D plots the estimated ranges of CWR for the CPA concentrations studied. Specifically, propylene glycol (PG, good glass forming tendency) and glycerol (poor glass forming tendency) were used as the boundaries for the estimated CWR (in orange) regions. The CWR of PG and glycerol were extrapolated from measurements using a rapid laser calorimetry method. In FIG. 3D, the warming rates of different approaches were marked, and the intersections of different CPA concentrations with those rates were identified, which represented various experimental conditions shown in FIG. 3E. Depending on whether the warming rate of the experimental condition is located above, within or below the CWR region, minimal (i.e., success), moderate (i.e., marginal success) or notable (i.e., failure) ice formation associated with the warming processes, respectively, was predicted. For instance, with 75% VS55, the warming rates for all Joule heating conditions (10 μs, 100 μs and 1 ms) are above the CWR region, indicating rewarming success. However, the warming rate for convective warming is within the CWR region, implying possible ice formation during rewarming. In addition, vitrification failure was noted for 25% VS55 and 50% VS55 CPAs during cooling.

The cell viability results in FIG. 3E illustrated the significance of rapid warming rates even under crystallized conditions. It was found that the viability was improved with faster warming rate when ice was expected during the cooling and/or warming phases. Even for 13.75% CPA, where ice crystallization is expected, 10 μs Joule heating provided 55.2 f 7.8% viability. In comparison, all the other warming conditions resulted in very low (<5%) viability for the 13.75% CPA conditions as indicated in FIGS. 3E-F. The estimated warming rate of 6.7×10⁸° C./min for 10 μs Joule heating represents the fastest warming rate achieved for cryopreservation rewarming (Table 1) (Jin B. et al. 2015 and Akiyama Y. et al. 2019), allowing the “rescue” of adherent cells with intracellular ice during rapid rewarming. In addition, with increased portion of ice formed during cooling (i.e., 25% VS55 vs. 50% VS55), fast warming rates may alleviate but not overwrite the ice injuries (FIG. 3E).

### Joule Heating for Drosophila Embryos Cryopreservation

Having validated the Joule heating cryopreservation approach in adherent cells, the performance in cryopreserving Drosophila embryos was examined, a more sophisticated and larger (i.e., 50 μm thickness) model system. Successful recovery of adult flies from cryopreserved embryos with a novel convective warming approach (providing rewarming rates on the order of 2.2×10⁵° C./min) using a nylon mesh and 39% EG+9% sorbitol as the CPA was previously reported. (Zhan, L. et al. 2021). To explore the capability to achieve cryopreservation with lower CPA concentrations in this model system, the post thaw survival rates between prior convective warming methods and Joule heating using the SS mesh was compared. A wildtype stock named WC1b (details in the Methods) was used. The woven SS mesh with 30 μm wire diameter and 38 μm aperture was used (FIG. 4A). Four CPA concentrations including 21%, 27%, 33% and 39 wt % EG, supplemented with 9 wt % sorbitol were tested. For CPA loading, embryos were equilibrated in 13% EG and then dehydrated with the higher CPA concentration mentioned above for 9 min on ice. Embryos were transferred to the SS mesh as a monolayer after CPA loading, with excess CPA solutions wicked away prior to plunge cooling. Images of embryos in liquid nitrogen were obtained to assess the ice formation after cooling (FIG. 4A). For 39% EG+9% sorbitol and 33% EG+9% sorbitol, it was found that most embryos appeared transparent, indicating vitrification success. Note that there are ice flakes present in the upper right corner of the image for 33% EG+9% sorbitol, but it is expected that these were condensed from the ambient air, not inside the embryos (FIG. 4A). As CPA concentrations decreased to 27% EG+9% sorbitol, the embryos started to appear hazy and some of them opaque (i.e., bottom area of the image) suggesting possible ice formation during cooling. In contrast, most of embryos turned opaque for 21% EG+9% sorbitol, suggesting the presence of ice.

Embryo rewarming through numerical simulation of Joule heating was investigated. The heat diffusion time across the embryo is ~1.3 ms. The dimensions of the SS mesh were 15 cm long and 1 cm wide, which provided a resistance of 1.4Ω and a RC time constant of 5.6 ms. To optimize the pulse width for Joule heating, the temperature profile and warming rate of the embryos for 0.1 ms, 1 ms and 10 ms pulses was simulated. The geometries of SS mesh and embryos used in the model are shown in FIG. 4B. Two representative embryos with different contact patterns with the heat source (i.e., the wires of SS mesh) were selected. The predicted SAR was 1×10¹³, 1.5×10¹² and 1.7×10¹¹ W/m³ for 0.1 ms, 1 ms and 10 ms Joule heating, respectively (FIGS. 9A-9C). The temperatures at the bottom of the embryo (point C, next to the SS mesh), middle of embryo (point B) and top of the embryo (point A) were studied as indicated in FIG. 4C. The temperature profiles illustrated an increasing temperature difference inside the embryos (i.e., point C vs point A) with decreasing pulse width (FIG. 8), similar to what was found earlier for the adherent cells (FIG. 3A). In addition, the warming rates for point A, B and C were 1.53×10⁸, 4.9×10⁶ and 3.8×10⁶° C./min for 0.1 ms pulse; 1.2×10⁷, 4.2×10⁶ and 3.3×10⁶° C./min for 1 ms pulse; 1.4×10⁶, 1.2×10⁶ and 1.1×10⁶° C./min for 10 ms pulse, respectively (FIG. 4C). The 1 ms pulse (comparable with t_{d}=1.3 ms) was selected to prioritize fast warming rates while avoiding large temperature gradient inside the embryo, compared with 0.1 ms and 10 ms pulses (FIG. 4C, FIGS. 9A-9C). As shown in FIG. 4D and FIGS. 9A-9C, the fraction of SS mesh that did not have a thermal load (i.e., no embryo contact, point E in FIG. 4B) has a higher temperature than the material with a contacting thermal load (i.e., embryo contact, point D in FIG. 4B). After the pulse ended, the SS mesh without embryo contact (i.e., Point E) continued to conduct heat toward the embryo, leading to a slightly higher warming rate around the periphery of the embryos (FIG. 4E). FIG. 4E further demonstrated the similar distribution of warming rates between embryos with different contact with the SS mesh, as the aperture size of SS mesh (38 μm) was relatively small compared with the dimensions of an embryo (180 μm×500 μm). This also suggests that mesh geometry will be an important factor to optimize for application in different biological system geometries.

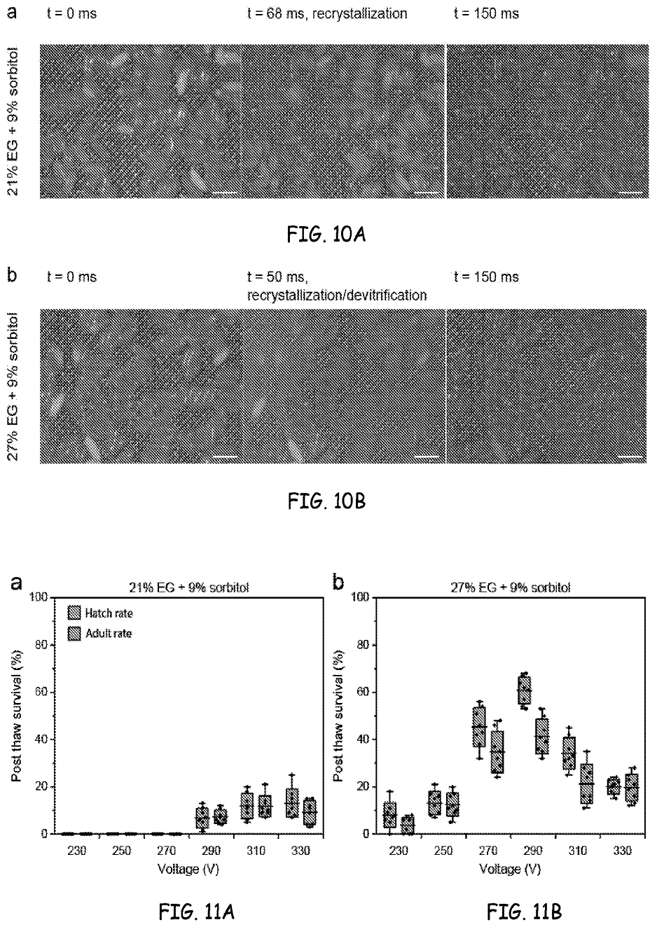

To experimentally monitor the warming performance of Joule heating, a high speed camera was employed to detect the ice formation during rewarming. Embryos loaded with different CPA concentrations were rewarmed with a 1 ms voltage pulse and recorded at 3000 frames per second (fps) under a dissecting microscope to capture more embryos. The grayscale intensity of embryos was analyzed using MATLAB. Higher intensity values were noted for white color (i.e., ice); lower intensity values (i.e., dark color) were noted when embryos were transparent/melted. For 27% EG+9% sorbitol, images of embryos before and after Joule heating are shown in FIG. 4F. Specifically, at t=0 ms, embryos were partially vitrified prior to Joule heating which was applied at t=~25 ms. At t=50 ms, most embryos turned hazy and a small amount of ice can be spotted, indicating that devitrification/recrystallization occurred during rewarming. The embryos appeared dark in color (i.e., similar to the SS mesh) at t=150 ms, suggesting complete melting. In FIG. 4G, the grayscale intensities (i.e., normalized to t=150 ms, when the embryos were completely melted) for all four CPA concentrations were displayed. For 21% EG+9% sorbitol and 27% EG+9% sorbitol, after pulse heating, the intensity first increased and then decreased, indicating ice formation during rewarming. A higher peak was observed for the 21% EG+9% sorbitol group, suggesting more ice formation as supported by the images in FIGS. 10A-10B. This is also expected as more ice was noted for the 21% EG+9% sorbitol group after cooling (FIG. 4A). In contrast, for 33% EG+9% sorbitol and 39% EG+9% sorbitol, the intensity dropped after the pulse heating, implying the absence of apparent ice formation. The intensity of the 39% EG+9% sorbitol group dropped faster than that of the 33% EG+9% sorbitol group, which might imply a faster warming rate. Indeed, it took ~25 ms for the intensity to drop to a constant level for the 39% EG+9% sorbitol group, which matched with the ~30 ms rewarming time (−196° C. to 0° C.) indicated by the numerical simulation (FIG. 4C).

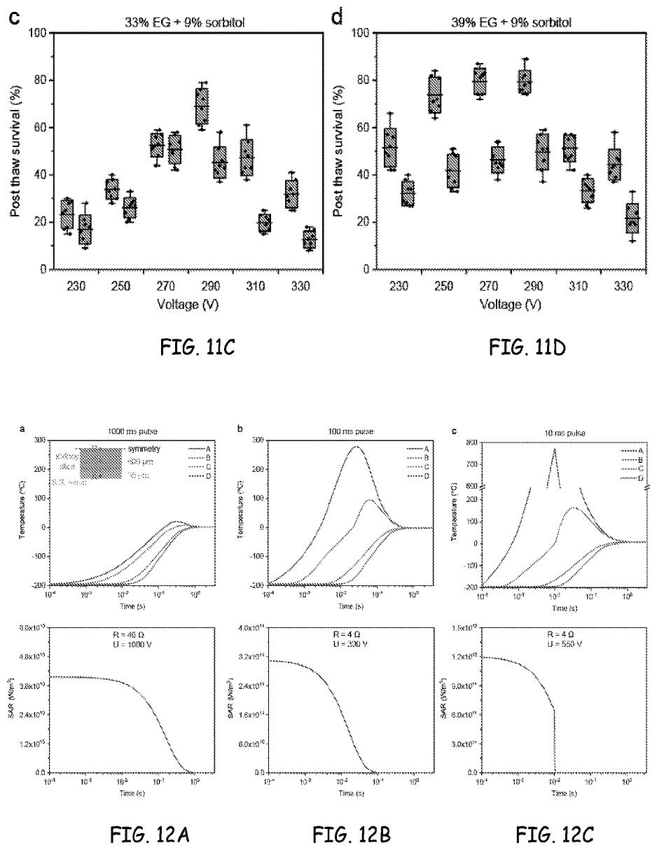

The embryo survival was evaluated for different CPA concentrations and rewarming methods. The survival rates were reported as hatch rate and adult rate which represent the embryo to larva survival and larva to adult survival, respectively. For CPA treated embryos (i.e., no cryopreservation), it was found that the hatch rate remained high (>90%) across different CPA concentrations. However, the adult rate decreased with increasing CPA concentrations, suggesting CPA toxicity affected the larvae to adult development. For rewarming, to optimize the voltage for Joule heating, we simulated the final temperature of the embryos assuming ice-free rewarming for simplicity. FIG. 4I indicated that voltages from 250V to 290V provided a final temperature ranging from −5° C. to 20° C. In addition, the survival rates are displayed in FIG. 4J and FIGS. 11A-11D for voltages from 230 V to 330 V at different CPA concentrations. The survival rates first increased then decreased with increasing voltages, transitioning from underheating to good warming and eventually to overheating. For the 39% EG group, no visible lethal ice was recorded during rewarming by 1 ms pulse Joule heating (FIG. 4G) and high viability was observed (FIG. 4k), suggesting the temperature gradient during rewarming (FIG. 10B) was tolerable in the Drosophila embryos. For the 27% EG+9% sorbitol group, although ice was observed during the rewarming (290V, 1 ms pulse) as depicted in FIG. 4F, it was shown that 60.8±5.3% embryos hatched into larvae and 41.3±6.9% larvae developed into adults. In contrast, for the 21% EG+9% sorbitol group where more ice was identified during cooling (FIG. 4A), it was found that the hatch rate and adult rate were 7.6±3.5% and 6.7±2.1%, respectively. The post thaw survival rates were compared between convective warming and Joule heating, as shown in FIG. 4K. When 39% EG+9% sorbitol was used, the convective warming produced similar survival with Joule heating, suggesting that the convective warming rate may have already surpassed the CWR. Importantly, Joule heating demonstrated higher survival than convective warming at lower CPA concentrations in the model system, which provides valuable opportunities for species less tolerable to the toxicity of high CPA concentrations.

Notably, we showed that certain small amounts of ice formation during rewarming are not lethal when rapid rewarming is applied. Specifically, Drosophila embryos treated with 27% EG+9% sorbitol were partially vitrified after cooling, rewarmed with 1 ms pulse at ~4.2×10⁶° C./min and recorded at 3000 fps. Ice formation was observed after the voltage pulse and lasted ~30 ms inside the embryos (FIG. 4F, 4G). We demonstrated that 60.8±5.3% embryos hatched into larvae and 41.3±6.9% larvae developed into adults. In comparison, with the similar warming rate, the survival decreased sharply when a lower CPA concentration (21% EG+9% sorbitol) was used and more ice was observed during rewarming. The amount of tolerable ice will likely depend greatly on the biological system and where the ice forms in the system.

### Joule Heating for Kidney Slices Cryopreservation

With the successful implementation in sub-millimeter systems including adherent cells and Drosophila embryos, Joule heating for cryopreservation of 1.2 mm thick rat kidney slices (diameter=6 mm) was tested as a model system. SS mesh was used to sandwich the kidney slice on each side to provide heating from both the top and bottom surfaces (FIG. 5A). Therefore, the characteristic heat diffusion length was reduced to 0.6 mm and led to a heat diffusion time of 180 ms. Numerical simulation was employed to optimize the pulse width (10 ms, 100 ms and 1 s) for Joule heating. Half of the kidney slice was modelled considering the expected planar symmetry in the system (FIG. 5B). The predicted SAR was 1.2×10¹², 3×10¹¹ and 4×10¹⁰ W/m³ for 10 ms, 100 ms and 1 s, respectively (FIGS. 12A-12C). In FIG. 5B, the center of the SS mesh (point A) and the bottom (point B), middle (point C) and top (point D) locations of the kidney slice were selected for temperature and warming rate comparison. The temperature profiles of points A-D are displayed for different pulse widths in FIGS. 12A-12C. The temperature difference inside the kidney slice increased with decreasing pulse width. The warming rates for points B, C, D were 1.6×10⁶, 1.1×10⁵ and 7×10⁴° C./min for 10 ms pulse; 9×10⁵, 9×10⁴ and 6.2×10⁴° C./min for 100 ms pulse; and 9.8×10⁴, 4.5×10³ and 3.8×10³° C./min for 1 s pulse, respectively (FIG. 5D). Based on this analysis, a 100 ms pulse width was selected to investigate further in the experiments. The warming rate across the thickness of the kidney slice is plotted in FIG. 5B. The temperature profile shown in FIG. 5C illustrates that the entire kidney slices took ~500 ms to reach an equilibrated final temperature while the pulse was 100 ms. The modelling suggests that the surface of the kidney slice (i.e., point B in FIG. 5C) could reach 40×94° C. for a short period of time (30 ms to 170 ms) during rewarming, resulting in negligible decrease in viability (i.e., 3.2%) due to thermally induced injury as estimated by the Arrhenius model (details in Methods) or increased CPA toxicity, which is expected at higher temperatures.

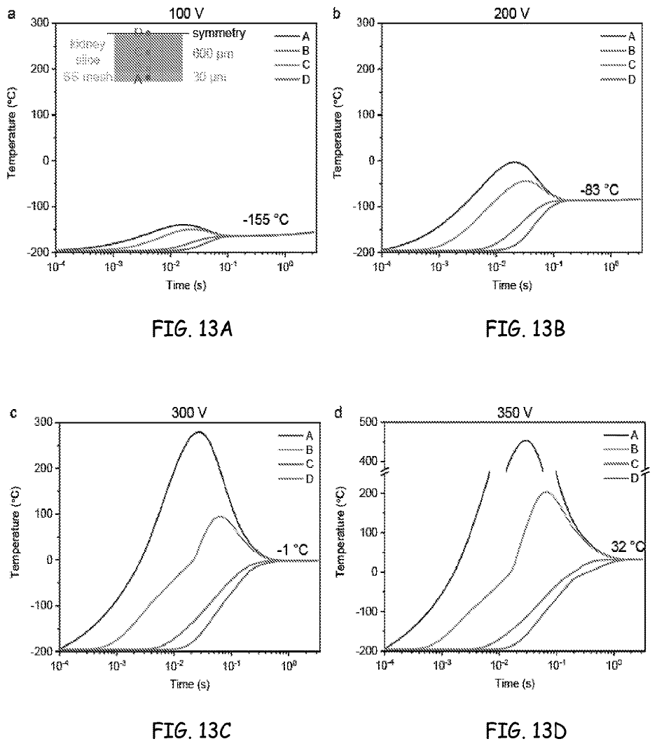

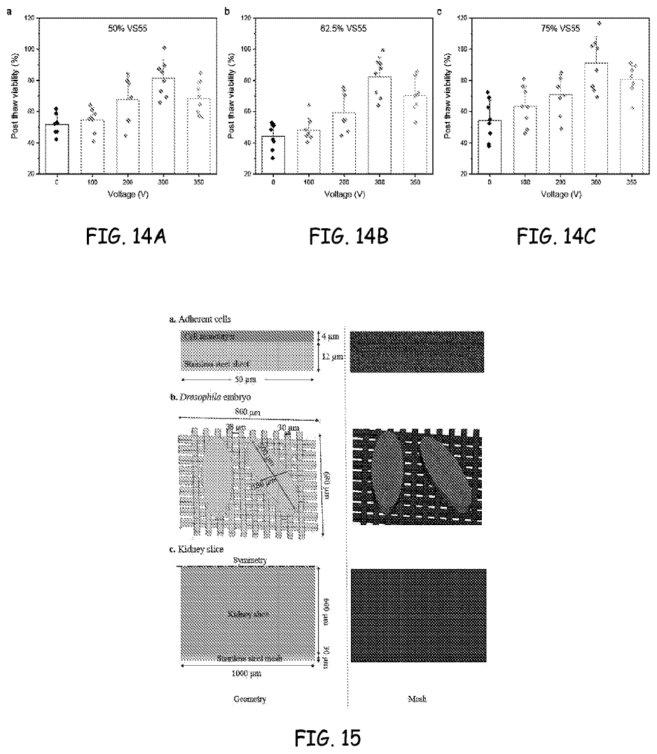

The Joule heating voltage was optimized using modeling and viability assessments. CPA loading was performed in a step-wise fashion (10 min each step) on ice by passive diffusion to minimize osmotic shock. After loading, the CPA concentration inside the kidney slice was modelled and is shown in FIG. 5E. Due to the slow diffusion mechanism, the CPA concentration is expected to decrease towards the middle plane of the kidney slices. For example, with 75% VS55, the CPA concentration loaded at the middle plane is 11.7 wt %, which further dropped to 6.2 wt % for 50% VS55. To ensure close contact for effective rewarming, a customized plastic tweezer with a 1.2 mm gap at the tip was used to hold the sandwich of the SS mesh/kidney slice during Joule heating. AlamarBlue was used to obtain fluorescence readings from the same kidney slice before and after cryopreservation. The viability was evaluated as the ratio of those two readings. For all three CPA concentrations, the viability was similar after CPA loading/unloading as presented in FIG. 5F. To optimize the voltage for Joule heating, the temperature profile was modelled. The predicted final temperature of the kidney slice after Joule heating increased from −155° C., −83° C., −1° C. to 32° C. for voltages of 100 V, 200 V, 300 V and 350 V, respectively (FIGS. 13A-13D). In addition, the viability was examined and showed that as the voltage increased, the viability increased until 300 V and dropped for 350 V (FIG. 5G and FIGS. 14A-14C). The decrease of viability at 350 V could be attributed to the overheating of the kidney slice surfaces (i.e., next to the SS mesh, FIG. 13D) and/or increased CPA toxicity at the higher final temperature (i.e., 32° C.). CPA exposure during cryopreservation protocols is typically conducted below at least 4° C., as toxicity has generally been shown to increase substantially at higher temperatures.

The viability between conventional convective warming and Joule heating was compared including different dilutions (50%, 62.5%, 75%) of VS55 as the CPA. For the convective warming group, the CPA loaded kidney slices were placed in a 2 ml cryovial which was plunged into liquid nitrogen for cooling and convectively rewarmed using a water bath as reported in the literature. The convective warming rate was approximately 100~200° C./min. The post thaw viabilities for 50% VS55, 62.5% VS55 and 75% VS55 were 42.1±4.8%, 50.3±6.8% and 52.9±4.5% using convective warming and 81.4±11%, 82.3±11.8% and 91.1±15.9% using Joule heating, respectively (FIG. 5H). In addition, to examine the gross structural morphology, H&E histological staining was performed for the conditions including a positive control (i.e., no treatment), optimal Joule heating (300V), air warming (i.e., negative control) and convective warming using 75% VS55 loaded kidney slices (FIG. 5I). For the Joule heating group, intact structures of glomerulus can be identified, and the Bowman's space was similar to the positive control group. In contrast, the convective warming groups showed disrupted glomerulus and some proximal tubules were damaged. Notable architectural disruption of the glomerulus and pale-staining were observed for the air warming group. Overall, the viability and histological results demonstrated that Joule heating showed great promise in improving cryopreservation of millimeter scale thickness tissues using low CPA concentrations.

### Physical Limits of Joule Heating Platform Technique

The design principles and physical limits of Joule heating for rewarming of biosystems with different heat diffusion lengths are summarized in FIGS. 6A-6C. To design Joule heating for rapid rewarming of biosystems with minimal thermal gradient, heat transfer scaling analysis was performed. Based on the thickness (h) of the biosystems, the heat diffusion time (t_{d}) across the biosystems was estimated assuming heat diffusivity (α) of 10⁻⁶ m²/s for 2 M glycerol laden tissues at sub-zero temperature. As demonstrated in the modelling results, short pulse widths lead to non-uniform warming and long pulse widths result in slow warming rates (FIGS. 3A, 4C, 5D, FIGS. 9A-9C, FIGS. 12A-12C). The optimal range of pulse width can be about tₚ~t_{d} to tₚ~10 t_{d} to achieve rapid and sufficiently uniform warming. The required input energy from Joule heating to rewarm the biosystems remains similar regardless of the pulse width, assuming heat loss/gain from the ambient is negligible for short pulse widths (<1 s). When t_{d}>>tₚ, non-uniform warming occurs, as the energy is delivered within a short time period, causing high temperature in the biosystem adjacent to the heat source, followed by heat diffusion to the entire biosystem after the voltage pulse ends. However, slow rewarming takes place for t_{d}<<tₚ. In this case, input energy is spread over a wide time window limiting the warming rate, but providing sufficient time for heat to diffuse across the entire biosystem on the same scale as it is generated. Cracking was not observed in any of the biosystems tested using optimal rewarming conditions, suggesting that a pulse width of tₚ~t_{d} to tₚ~10 t_{d} provided sufficiently uniform rewarming.

In addition, the achievable warming rate is limited by the heat diffusion length of the biosystem (FIG. 6B). The estimated overall warming rates can be calculated as 200° C./tₚ, where 200° C. is the temperature difference from liquid nitrogen (−196° C.) to 4° C. (assumed representative of typical rewarming conditions). For example, when tₚ=t_{d}, estimated warming rates for biosystems with 10 μm, 100 μm, 1 mm and 1 cm heat diffusion length are 2×10⁸, 2×10⁶, 2×10⁴ and 2×10²° C./min, respectively. Based on these estimated warming rates, the target CPA concentration inside the biosystems can be estimated to realize optimal cryopreservation (FIG. 6c). Two of the major failure modes of cryopreservation are CPA toxicity (i.e., CPA concentration too high) and ice crystallization (i.e., CPA concentration too low), assuming the biosystems can be successfully cryopreserved without cracking. Within the rapid and uniform warming region in FIG. 6A (i.e., t_{d}≤tₚ≤10 t_{d}), the upper and lower bounds of optimal CPA concentrations were estimated using a slower warming rate (tₚ=10 t_{d}) with glycerol (relatively high CWR) as the CPA, and a faster warming rate (tₚ=t_{d}) with PG (relatively low CWR) as the CPA, respectively. The estimated lowest CPA concentrations (blue line in FIG. 6C) required for biosystems with 10 μm, 100 μm, 1 mm and 1 cm heat diffusion length are 15%, 28%, 40% and 53 w/w %, respectively. In practice, the optimal CPA concentration could be increased to facilitate successful cryopreservation given that CPA toxicity can be minimized with optimized loading/removal protocols.

The complete disclosure of all patents, patent applications and publications, and electronically available material cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The description is not limited to the exact details shown and described, for variations obvious to one skilled in the art will be included within the description defined by the claims.

Although the present invention has been described with reference to preferred embodiments, workers skilled in the art will recognize that changes may be made in form and detail without departing from the spirit and scope of the invention.

1. A method of rewarming a cryopreserved biological composition comprising:
applying resistive heating to rewarm a cryopreserved biological composition, wherein the resistive heating comprises discharging a tunable voltage pulse from an electrical generator to an electrical conductor, wherein the generator is electrically connected to the conductor, wherein the cryopreserved biological composition comprises a cryopreserved biomaterial suspended in and/or perfused with a cryoprotective solution, wherein the cryopreserved biological composition is in thermal contact with the conductor, and wherein the voltage pulse is tuned to generate heat sufficient to rewarm across a heat diffusion length of the cryopreserved biomaterial with minimal biomaterial damage.

2. The method of claim 1, wherein the heat diffusion length is about the thickness of the biomaterial, is about half the thickness of the biomaterial, or is between about 1 μm and about 10 mm.

3. (canceled)

4. (canceled)

5. The method of claim 1, wherein the voltage pulse discharged from the generator is tuned to generate a minimum heating rate and a selected temperature change in the cryopreserved biomaterial.

6. The method of claim 5, wherein the method further comprises determining a voltage pulse width that generates rewarming above the minimum heating rate across the heat diffusion length of the biomaterial.

7. (canceled)

8. The method of claim 6, wherein the voltage pulse width is between about the heat diffusion time of the cryopreserved composition and about 10 times the heat diffusion time of the cryopreserved composition or wherein the voltage pulse width is between about 0.1 μs and about 10 seconds.

9. (canceled)

10. (canceled)

11. The method of claim 1, wherein the conductor comprises stainless steel, titanium, platinum, aluminum, zinc, gold, silver, nickel, iron, chromium, molybdenum, silicon, carbon, ceramic, doped plastic and combinations thereof.

12. The method of claim 1, wherein the conductor comprises a conductivity and a geometry, and wherein the conductivity and the geometry of the conductor are selected to generate a specific absorption rate (SAR) for rewarming the biomaterial at a heating rate to minimize biomaterial damage.

13. The method of claim 12, wherein the geometry of the conductor comprises a sheet, a mesh, a cylinder, a coil, a wound structure, a porous structure, a matrix structure, a channel and combinations thereof.

14. The method of claim 1, wherein the conductor is in a configuration adapted to generate a sufficiently uniform heating rate across the heat diffusion length to minimize damage to the biomaterial.

15. The method of claim 1, wherein an intermediate layer is between the conductor and the biomaterial.

16. The method of claim 1, wherein the biomaterials are selected from the group consisting of adherent cells, droplets or thin films of cell suspensions, protein suspensions, embryos, oocytes, larvae, organisms, tissue slices, biopsies, isolated tissues, engineered tissues, cell clusters, organoids and the like.

17. The method of claim 1, wherein the voltage pulse form is an exponentially decaying wave, sine wave, a rectangular wave, a triangular wave, a sawtooth waves or a square wave.

18. (canceled)

19. (canceled)

20. The method of claim 1, wherein the biomaterial damage is from ice crystallization causing damage to cellular and tissue structures or is from non-uniform warming leading to overheating and/or thermomechanical stresses in the biomaterial.

21-43. (canceled)

44. A system for rewarming a cryopreserved composition comprising: an electrical generator electrically connected to an electrical conductor, wherein the electrical conductor is configured for thermal contact with a cryopreserved composition comprising a biomaterial suspended in and/or perfused with a cryoprotective solution, wherein a discharge of a tunable voltage pulse from the electrical generator generates heat sufficient to rewarm across the heat diffusion length of the biomaterial with minimal biomaterial damage.

45. (canceled)

46. The system of claim 44, wherein the voltage pulse discharged from the generator is tuned to generate a minimum heating rate and a selected temperature change in the cryopreserved biomaterial sufficient to rewarm the cryopreserved biomaterial with minimal biomaterial damage.

47. The system of claim 46, wherein the voltage pulse comprises a voltage pulse width tuned to generate rewarming above the minimum heating rate across the heat diffusion length of the biomaterial.

48. (canceled)

49. The system of claim 47, wherein the voltage pulse width is between about the heat diffusion time of the cryopreserved composition and about 10 times the heat diffusion time of the cryopreserved composition or is between about 0.1 μs and about 10 seconds.

50. (canceled)

51. (canceled)

52. The system of claim 44, wherein the biomaterials are selected from the group consisting of adherent cells, droplets or thin films of cell suspensions, protein suspensions, embryos, oocytes, larvae, organisms, tissue slices, biopsies, isolated tissues, engineered tissues, cell clusters, organoids and the like.

53. The system of claim 44, wherein the heat diffusion length is between lpm and about 10 mm.

54. (canceled)

55. (canceled)

56. The system of claim 44, wherein the conductor comprises a conductivity and a geometry, and wherein the conductivity and the geometry of the conductor are selected to generate a specific absorption rate (SAR) for rewarming the biomaterial at a heating rate to minimize biomaterial damage.

57. The system of claim 56, wherein the geometry of the conductor comprises a sheet, a mesh, a cylinder, a coil, a wound structure, a porous structure, a matrix structure, a channel and combinations thereof.

58. (canceled)

59. The system of claim 44, wherein an intermediate layer is between the conductor and the biomaterial.

60. The system of claim 44, wherein the voltage pulse form is an exponentially decaying wave, sine wave, a rectangular wave, a triangular wave, a sawtooth wave or a square wave.

61. The system of claim 44, wherein the magnitude of the voltage pulse is between about 1V and 10,000V.

62. The system of claim 44, wherein the system is adapted for high throughput and/or larger size of the biomaterial.

63. (canceled)

64. (canceled)
